(19) United States
(12) Patent Application Publication
(10) Pub. No.: US 2009/0062624 A1
(43) Pub. Date: Mar. 5, 2009

(54) METHODS AND SYSTEMS OF DELIVERING A PROBABILITY OF A MEDICAL CONDITION

(76) Inventor: Thomas Neville, Incline Village, NV (US)

Correspondence Address: WILSON SONSINI GOODRICH & ROSATI, 650 PAGE MILL ROAD, PALO ALTO, CA, 94304-1050, US

(21) Appl. No.: 12/109,832

(22) Filed: Apr. 25, 2008

Related U.S. Application Data
(60) Provisional application No. 60/914,125, filed on Apr. 26, 2007.

(51) Int. Cl. A61B 5/00; G06Q 50/00

(52) U.S. Cl. 600/300; 705/2

(57) ABSTRACT

Methods and systems for delivering a probability that a subject has a medical condition are disclosed herein. The methods comprise calculating the probability of a medical condition using biomarker values and the rate of change of the biomarker values over time. In most embodiments, the methods comprise relations and calculations that require computer systems to execute the methods of the invention. Systems of the invention may include computer systems, as well as medical systems, such as biomarker assays and courses of medical action.

### CROSS-REFERENCE

This application claims the benefit of U.S. Provisional Application No. 60/914,125, filed Apr. 26, 2007, which application is incorporated herein by reference in their entirety.

### BACKGROUND OF THE INVENTION

In the field of medicine there is increasing emphasis on: health, disease prevention and early detection and treatment; avoiding unnecessary treatment; choosing the optimal timing of the best treatment based on medical evidence; and avoiding invasive and costly procedures like biopsies.

Significant investments are being made to accelerate discovery and use of biomarkers that effectively detect a medical condition. However, many of the new biomarkers are not adequately effective based on the results of a single test.

The use of screening blood tests for multiple markers is becoming more prevalent and cost effective. New techniques reduce the cost of specific tests. One blood draw to test many markers to screen for a plurality of medical conditions at a single time reduces the overall cost of screening. The incremental cost of additional tests decreases once blood is drawn for another test. Blood can be stored for later testing if needed for specific conditions in order to reduce costs of establishing biomarker data over time.

There is a need in the art for method and systems that can process large quantities of biomarker test results over time to derive actionable information from the tests. Often, biomarker values, such as concentrations, are not enough to discern the medical condition of a subject. For example, individuals with a high body mass index (BMI) may dilute the concentration of certain markers and adjustments to the results are needed. Marker concentrations can vary substantially among healthy individuals, whereas the concentrations over time and the rate of change may provide more valuable information. There is a need for a data processing method that can create actionable information from one or a plurality of biomarker values, either from an individual test of a plurality of tests over time.

### SUMMARY OF THE INVENTION

In general, in one aspect, a method of delivering a probability that a subject has a medical condition to a user is provided including a) calculating a posterior probability that a subject has a medical condition, wherein said subject has a biomarker trend, wherein said trend is formed by values corresponding to a biomarker for said medical condition obtained at least two different times from said subject, by relating: i) a probability of observing said biomarker trend for an individual with said medical condition; ii) a probability of observing said biomarker trend for an individual without said medical condition; and iii) a prior probability that said subject has said medical condition; and b) delivering said posterior probability to a user with an output device.

In another aspect, a method of delivering a probability that a subject has a medical condition to a user is provided including a) calculating a posterior probability that a subject has a medical condition, wherein said subject has a biomarker value for said medical condition, by relating: i) a probability of observing said biomarker value for an individual with said medical condition, ii) a probability of observing said biomarker value for an individual without said medical condition; and iii) a prior probability that said subject has said medical condition; and b) delivering said posterior probability to a user with an output device.

In general, in yet another aspect, a method of delivering a probability that a subject has a medical condition to a user is provided including a) calculating a posterior probability that a subject has a medical condition, wherein said subject has a first biomarker value and a second biomarker value for said medical condition, wherein said second biomarker value is obtained after said first biomarker, by relating: i) a probability of observing said second biomarker value for an individual with said medical condition; ii) a probability of observing said second biomarker value for an individual without said medical condition; iii) a probability of observing a biomarker rate of change for an individual with said medical condition, wherein said biomarker rate of change is the difference of biomarker values over time; iv) a probability of observing said biomarker rate of change for an individual without said medical condition; and v) a prior probability that said subject has said medical condition; and b) delivering said posterior probability to a user with an output device.

In an embodiment, the probability of observing said biomarker trend for an individual with said medical condition can be calculated by comparing said biomarker trend to a historical probability distribution of historical biomarker trends of a population with said medical condition. The probability of observing said biomarker trend for an individual without said medical condition can be calculated by comparing said biomarker trend to a historical probability distribution of historical biomarker trends of a population without said medical condition. The probability of observing said biomarker value for an individual with said medical condition can be calculated by comparing said biomarker value to a historical probability distribution of historical biomarker values of a population with said medical condition. The probability of observing said biomarker value for an individual without said medical condition is calculated by comparing said biomarker value to a historical probability distribution of historical biomarker values of a population without said medical condition.

In an embodiment, a biomarker rate of change of change is a trend. In another embodiment, a biomarker rate of change is the slope of a trend. In an embodiment, trend can be used interchangeably with the slope or derivative or velocity of a line or connector between two values.

A prior probability can be calculated by comparing a profile of said subject to historical probabilities of said medical condition in an individual of a population.

In an embodiment, the methods can further include biomarker values from a second biomarker corresponding to said medical condition.

In an embodiment, a medical condition is cancer, such as prostate cancer. The biomarker can be fPSA or PSA.

The methods can further include removing a biomarker value from said biomarker trend that has a value outside a tolerance. The tolerance can be determined by a historical biomarker trend representing said individual of a population with or without said medical condition. The tolerance can be set by said user. The tolerance can be set automatically.

Calculating a posterior probability that a subject has a medical condition can include, for example, at least one Monte Carlo simulation. Calculating a posterior probability that a subject has a medical condition can be carried out by a computer system. The computer system can include, for example, a Monte Carlo calculation engine. The user can be selected from the group including the following: said subject, a medical professional, a clinical trial monitor, and a computer system.

In general, in yet another aspect, a method of taking a course of medical action by a user is provided including initiating a course of medical action based on a posterior probability delivered from an output device to said user.

The course of medical action can be delivering medical treatment to said subject. The medical treatment can be selected from a group consisting of the following: a pharmaceutical, surgery, organ resection, and radiation therapy. The pharmaceutical can include, for example, a chemotherapeutic compound for cancer therapy. The course of medical action can include, for example, administration of medical tests, medical imaging of said subject, setting a specific time for delivering medical treatment, a biopsy, and a consultation with a medical professional.

The course of medical action can include, for example, repeating a method described above.

A method can further include diagnosing the medical condition of the subject by said user with said posterior probability from said output device.

In general, in yet another aspect, a computer readable medium is provided including computer readable instructions, wherein the computer readable instructions instruct a processor to execute step a) of the methods described above. The instructions can operate in a software runtime environment.

In general, in yet another aspect, a data signal is provided that can be transmitted using a network, wherein the data signal includes said posterior probability calculated in step a) of the methods described above. The data signal can further include packetized data that is transmitted through a carrier wave across the network.

In general, in yet another aspect, a medical information system for delivering a probability of a medical condition of a subject to a user is provided including: a) an input device for obtaining biomarker values corresponding to a biomarker for a medical condition at least two different times from said subject, wherein said biomarker values form a biomarker trend; b) a processor in communication with said input device, wherein said processor uses said biomarker trend to calculate a posterior probability of said subject having said medical condition; and c) a storage unit in communication with at least one of the input device and the processor, wherein said storage unit includes at least one database including said biomarker values, said posterior probability, or a prior probability of said subject having said medical condition; and d) an output device in communication with at least one of said processor and said storage unit, wherein said output device transmits said posterior probability to a user.

The input device can be a graphical user interface of a webpage. The input device can be an electronic medical record. In an embodiment, a medical condition is prostate cancer. The biomarker can be PSA or fPSA.

In an embodiment, a processor and a storage unit can be part of a computer server. The processor can calculate a posterior probability that a subject has a medical condition by relating: a) a probability of observing said biomarker trend for an individual with said medical condition; b) a probability of observing said biomarker trend for an individual without said medical condition; and c) a prior probability that said subject has said medical condition.

An output device can be selected from a group including the following: a graphical user interface of a webpage, a print-out, and an email. The communication can be wireless communication.

In another embodiment, a system of the invention can further include a medical test for testing said subject for said medical condition. The medical test can be a PSA assay. In yet another embodiment, a system can further include a medical treatment for treating said subject for said medical condition. The medical treatment can be selected from a group including the following: a pharmaceutical, surgery, organ resection, and radiation therapy.

In general, in yet another aspect, a method of delivering a probability of a medical condition of a subject to a user is provided including a) collecting biomarker values from a subject corresponding to a biomarker for a medical condition at least two different times, wherein the biomarker values at the at least two different times form a biomarker trend; b) exporting said biomarker trend for analysis, wherein said analysis includes: calculating a posterior probability that a subject has a medical condition by relating, i) a probability of observing said biomarker trend for an individual with said medical condition; ii) a probability of observing said biomarker trend for an individual without said medical condition; and iii) a prior probability that said subject has said medical condition; c) importing the results of said analysis to an output device; and d) delivering said posterior probability to a user with said output device.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an example of a dynamic screening system.

FIG. 2 is a flowchart for a dynamic screening system.

FIG. 3 depicts long-term probabilities processing.

FIG. 4 illustrates a flow chart for utilizing personal probability distributions of progressing cancer.

FIGS. 5-7 depict flow charts demonstrating methods of calculating personal probability distributions using Monte Carlo methods.

FIGS. 8-10 show a Monte Carlo process for generating outcomes from a number of probability distributions.

FIG. 11 shows 100 possible buckets of possible results when two dimensions are divided into ten segments.

FIG. 12 depicts 10,000 possible buckets of possible results when three dimensions are divided into ten segments.

FIG. 13 depicts the number of Monte Carlo iterations required to create a reasonably stable distribution.

FIG. 14 depicts a bucket of concern defined by a range of PSA and PSAV results around observed trend results.

FIG. 15 illustrates a small cube inside a large cube depicting a hypercube bucket of concern defined by a range biomarker results

FIG. 16 depicts methods for reducing the number of calculations by focusing on a bucket of concern are disclosed below.

FIG. 17 shows a four dimensional frequency generator for a no cancer case.

FIG. 18 shows a Monte Carlo process for generating no cancer PSA outcomes from a number of probability distributions.

FIG. 19 demonstrates total calculation time can be reduced by constraining the range of values used to calculate PSA to the combinations of values that are likely to result in trend PSA values that are within range of a target value.

FIGS. 20-22 show a Monte Carlo process for generating outcomes from a number of probability distributions.

FIG. 23 shows a four dimensional frequency generator for each year of cancer plus a no cancer case.

FIGS. 24-27 depict flow charts showing a Monte Carlo process for generating outcomes from a number of probability distributions.

FIG. 28 depicts a dynamic screening custom content system,

FIG. 29 demonstrates a flow chart where two types of feedback learning can improve the method over time.

FIG. 30 illustrates an exemplary feedback process where information about outcomes can be fed back to individual screening history and to all screening history for analysis of groups of individuals.

FIG. 31 illustrates an exemplary computer system of the invention comprising a plurality of graphical user interfaces, a front end server comprising databases, and a back end server capable of performing calculations of probabilities.

FIG. 32 illustrates an exemplary method of delivering a probability that a subject has a medical condition to a user and using the probability to take a course of medical action.

FIGS. 33-34 illustrate exemplary courses of events related to a method or system of the invention.

FIG. 35 shows an example using a method of the invention where AUCs were highest for younger men and declined with age.

FIG. 36 demonstrates an example method wherein AUCs increased with mean tumor volume but did not vary substantially by Gleason group, except for the smallest tumor volumes.

FIG. 37 demonstrates in an example that AUCs increased as the false positive rejection percentage.

### DETAILED DESCRIPTION OF THE INVENTION

Methods and systems for delivering a probability that a subject has a medical condition are disclosed herein. In most embodiments, the methods comprise relations and calculations that require computer systems to execute the methods of the invention. Systems of the invention may include computer systems, as well as medical systems, such as biomarker assays and courses of medical action.

In an aspect, computer-implemented personalized probabilities determination systems and methods for use in integrated health systems and methods are disclosed herein related to organs of the human body and to cancer.

For example, a system and method is disclosed herein for estimating trends in biomarkers and calculating the probability of medical conditions of one or more organs of the human body. This could be used for any condition of any organ of the human body. An example application of the male prostate with a focus on progressing prostate cancer is disclosed as an example here without limitation.

A system to perform the Bayes calculation of the probability of progressing cancer can be configured with the following components: prior probabilities of cancer at various stages of progression; probability of the observation of various biomarker trends conditional on no progressing cancer; and probability of the observation of various biomarker trends conditional on cancer at various stages of progression.

In general, in one aspect, a method of delivering a probability that a subject has a medical condition to a user is provided including a) calculating a posterior probability that a subject has a medical condition, wherein said subject has a biomarker trend, wherein said trend is formed by values corresponding to a biomarker for said medical condition obtained at least two different times from said subject, by relating, i) a probability of observing said biomarker trend for an individual with said medical condition; ii) a probability of observing said biomarker trend for an individual without said medical condition; and iii) a prior probability that said subject has said medical condition; and b) delivering said posterior probability to a user with an output device.

In an embodiment, a medical condition is any condition of a subject relating to a particular disease. For example, a medical condition can be progressing cancer. In another embodiment, a medical condition is infection. In another embodiment, a medical condition sepsis. A medical condition can be any condition of a subject determined by a medical professional.

In another aspect, a method of delivering a probability that a subject has a medical condition to a user is provided including a) calculating a posterior probability that a subject has a medical condition, wherein said subject has a biomarker value for said medical condition, by relating: i) a probability of observing said biomarker value for an individual with said medical condition, ii) a probability of observing said biomarker value for an individual without said medical condition; and iii) a prior probability that said subject has said medical condition; and b) delivering said posterior probability to a user with an output device.

In an embodiment, the probability of observing said biomarker trend for an individual with said medical condition can be calculated by comparing said biomarker trend to a historical probability distribution of historical biomarker trends of a population with said medical condition. The probability of observing said biomarker trend for an individual without said medical condition can be calculated by comparing said biomarker trend to a historical probability distribution of historical biomarker trends of a population without said medical condition. The probability of observing said biomarker value for an individual with said medical condition can be calculated by comparing said biomarker value to a historical probability distribution of historical biomarker values of a population with said medical condition. The probability of observing said biomarker value for an individual without said medical condition is calculated by comparing said biomarker value to a historical probability distribution of historical biomarker values of a population without said medical condition.

In an embodiment, a biomarker rate of change of change is a trend. In another embodiment, a biomarker rate of change is the slope of a trend. In an embodiment, trend can be used interchangeably with the slope or derivative or velocity of a line or connector between two values.

In an embodiment, the probability of observing said biomarker trend for an individual with said medical condition can be calculated by comparing said biomarker trend to a historical probability distribution of historical biomarker trends of a population with said medical condition. The probability of observing said biomarker trend for an individual without said medical condition can be calculated by comparing said biomarker trend to a historical probability distribution of historical biomarker trends of a population without said medical condition. The probability of observing said biomarker value for an individual with said medical condition can be calculated by comparing said biomarker value to a historical probability distribution of historical biomarker values of a population with said medical condition. The probability of observing said biomarker value for an individual without said medical condition is calculated by comparing said biomarker value to a historical probability distribution of historical biomarker values of a population without said medical condition.

In an embodiment, a biomarker value is a value obtained from a biomarker belonging to a subject. For example, a biomarker value can be a concentration or any other measure or unit as would be obtained from a biomarker assay or test. A value of a biomarker obtained from a subject can be of a measure or units as would be obvious to one skilled in the art.

In an embodiment, a biomarker trend is at least two values of the same biomarker from different time points.

In an embodiment, an individual with said medical condition is an individual from a population of subjects with the medical condition. In an embodiment, an individual without said medical condition is an individual from a population of subjects without the medical condition.

In an embodiment, historical biomarker values are biomarker values from historical or previous studies that relate values of a biomarker to a medical condition. For example, historical biomarker values can be the results of a clinical study, for example a study that shows PSA is biomarker for prostate cancer.

In an embodiment, a historical probability distribution is a probability distribution of how historical biomarker trends or values relate to a medical condition in a population of subjects with the medical condition. In another embodiment, historical probability distribution is the frequency at which the values or trends predict to a medical condition in a population of subjects with the medical condition.

In an embodiment, a prior probability is any probability that a subject has a medical condition before carrying out a method of the invention. For example, the prior probability can be calculated from the profile of subject, such as the subject's sex, age, weight, and race. A profile of a subject may be associated with the medical condition based on empirical evidence from historical studies, wherein the profile then has a probability of being associated with the medical condition. In an alternate embodiment, a prior probability is randomly assigned, In another embodiment, a prior probability is based on the posterior probability delivered from a method of the invention. In yet another embodiment, a prior probability is determined by a medical professional or a series of medical tests. Any other method of determining a prior probability of the subject having the medical condition can be used as would be obvious to a medical professional, statistician, computer, or one skilled in the art.

A prior probability can be calculated by comparing a profile of said subject to historical probabilities of said medical condition in an individual of a population.

In an embodiment, the methods can further include biomarker values from a second biomarker corresponding to said medical condition.

In an embodiment, a medical condition is cancer, such as prostate cancer. The biomarker can be fPSA or PSA.

The methods can further include removing a biomarker value from said biomarker trend that has a value outside a tolerance. The tolerance can be determined by a historical biomarker trend representing said individual of a population with or without said medical condition. The tolerance can be set by said user. The tolerance can be set automatically.

Calculating a posterior probability that a subject has a medical condition can include, for example, at least one Monte Carlo simulation. Calculating a posterior probability that a subject has a medical condition can be carried out by a computer system. The computer system can include, for example, a Monte Carlo calculation engine. The user can be selected from the group including the following: said subject, a medical professional, a clinical trial monitor, and a computer system.

A system can be configured for generating one or both of two categories of probabilities for an individual man with specific observed biomarker trends and corresponding measurement uncertainty in those trends.

Consider a man concerned about prostate cancer with a series of PSA and free PSA biomarker results from blood tests. Trends can be estimated for each biomarker and analyzed using methods previously disclosed. The results might be: trend PSA (3.0±0.4), trend PSA velocity (0.40±0.20), trend free PSA % (17.0±2.0%), and trend free PSA velocity % (6.0%±3.0%), where trend PSA velocity is the annual rate of change in trend PSA; trend free PSA % is trend free PSA divided by trend PSA; and trend free PSA velocity % is trend free PSA velocity divided by trend PSA velocity.

Other information about the man may be available including, but not limited to, age, measurement of prostate volume in some cases, and other factors that may affect the conditional probabilities.

Typically, no highly specific conditional distributions can be estimated directly from available population data.

In an embodiment, the method starts by creating personalized biologic probability models of: (a) no cancer conditions of the prostate: healthy and volume growth; (b) cancer at various stages of progression; and (c) combined models of no cancer conditions and various stages of cancer progression. Those models are then combined with trend uncertainty models to create an overall multi-dimensional distribution or part of the distribution relevant to the specific trend results. The distributions are multi-dimensional in that trend values and trend velocities, or annual rates of change, are considered for at least one biomarker, such as PSA. The disclosed example describes a method for creating four dimensional distributions and probabilities for two biomarkers: PSA and free PSA. Higher dimensional distributions and probabilities may be needed when additional biomarkers are considered.

Monte Carlo methods are used to create four dimensional probability distributions for PSA, PSAV, fPSA % and fPSAV % from random draws from the probability distributions of the underlying biologic and trend uncertainty models. The calculation process can be time consuming and slow the response for online users. The complexity and time of calculation can increase exponentially as additional biomarkers become available and are incorporated into the method. Therefore, efficient methods of calculating the probabilities can be beneficial.

In an embodiment a focuses on the probabilities of the observed trend values rather than very much larger four dimensional probability distributions for PSA, PSAV, fPSA % and fPSAV % for the full range of possible outcomes. This approach reduces the amount of calculations necessary to calculate the personalized probabilities needed for the Bayes calculations. The reduction is achieved in practice using a hierarchical triage approach that aborts a Monte Carlo iteration as soon as one of the values falls outside the target range for first PSA, then PSAV, then fPSA % and finally fPSAV %.

A dynamic screening system can help men and their doctors screen for progressing cancer, long-term conditions and short-term conditions. It can provide early warning of progressing cancer while reducing the probability of unnecessary treatment and side effects. The results can be useful input for the timing of treatment or course of medical action. The prostate is the organ of the body used in the many of the examples and conditions used as examples are progressing prostate cancer, prostate volume growth caused by Benign Prostatic Hyperplasia (BPH) and infections of the prostate. Both PSA and free PSA tests can be used for screening. Other tests may supplement them or replace them.

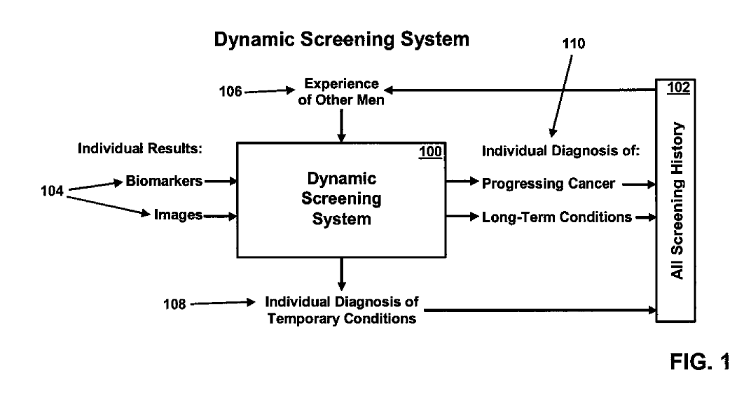

The flow chart on FIG. 1 provides a high level overview of the dynamic screening system. For one person, biomarker and image results are input on the left (104). For the prostate, they are PSA and free PSA test results and ultrasound measurements of prostate volume. The experience of other men is input from the top (106). A diagnosis of temporary conditions comes out the bottom (108). For the prostate, an infection is the most common and serious temporary condition. Diagnoses of progressing cancer and long-term conditions (volume growth due to BPH for the prostate) are output on the right (110). All output becomes part of all screening history (102) and is fed back as the experience of other men to increase the power of dynamic screening (106).

A man or his doctor can register him as a new user and completes a subject profile for him. Using the dynamic screening system, the man follows suggestions about the type and timing of primary and secondary screening tests. Typically the system can recommend a baseline prostate volume study and annual PSA and free PSA tests. Free PSA tests are currently recommended; however, other tests may be recommended in the future in conjunction with free PSA or to substitute for it. Tests results can be entered into the system for analysis and guidance. Steadily increasing PSA due to prostate enlargement from BPH, if rapid enough, can lead the system to suggest periodic prostate volume measurements to define the rate of growth. Tests results can be entered into the system for analysis and guidance.

The dynamic screening system may recognize the false alarms caused by infection and other temporary conditions, provide calming perspective, suggest new PSA and free PSA tests after the infection or condition has passed, and analyze the results of new tests. The dynamic screening system may also recognize early warning of possible cancer progression and suggest additional confirmation tests. Confirmation tests may include other components of PSA such as pro PSA and any other useful new markers developed in the future. In addition, a new prostate volume study may be suggested, perhaps using more expensive technology if rapid prostate enlargement is a factor. A second round of confirmation tests can be suggested, for example six months after the first. Additional confirmation tests can be suggested until progression has been confirmed or rejected.

In an embodiment, the dynamic screening system confirms a high probability of progressing cancer when its calculation shows the probability is high enough to warrant consideration of biopsy and treatment

A timing system can calculate the optimal schedule for biopsy and treatment based on ongoing screening tests and the information entered in a subject profile. The man and his advisors can use the results to schedule a first biopsy and subsequent treatment. A man or his doctor can also provide follow up information for the system to analyze and incorporate for use by other men.

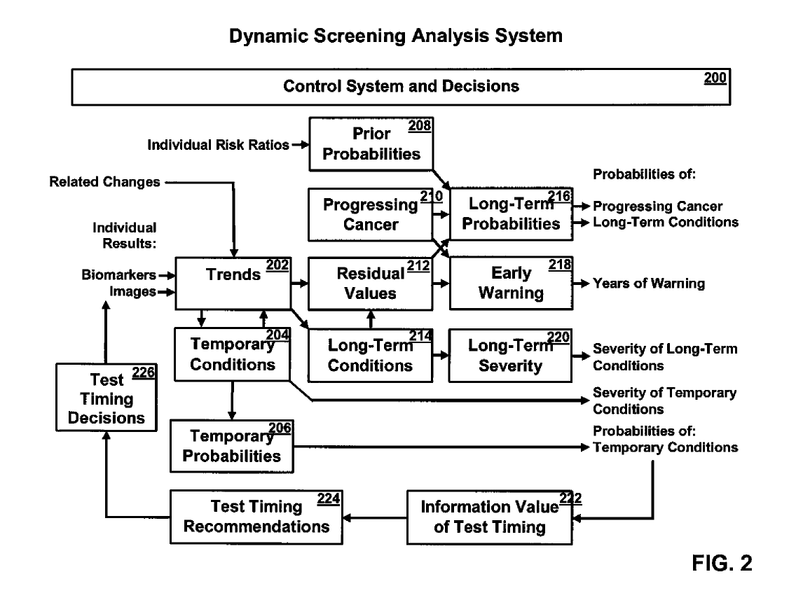

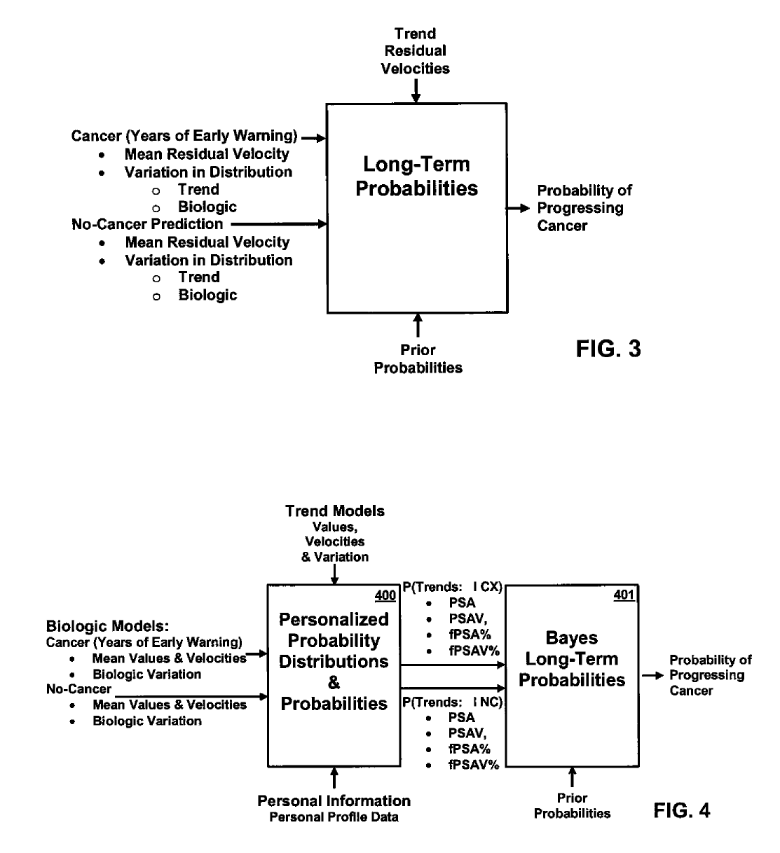

A long-term probabilities module (216) on FIG. 2 estimates the probabilities of one or more long-term conditions, such as progressing cancer or prostate volume growth. FIG. 3 shows an example of the high level inputs and outputs for estimating the probability of progressing cancer. Prior probabilities are the starting point and come from module 208 on FIG. 2. Trend residual velocities come from module 212 on FIG. 2. Velocities and trends may be used in other embodiments. The long-term probabilities module on FIG. 3 adjusts the prior probabilities of progressing cancer based on how the trend residual velocities compare with patterns for progressing cancer and the predicted values for no cancer. A variety of methods can be used to estimate the probability, including Bayesian and simulation methods. The process can involve a variety of cancer stages, characterized by years of early warning, for example measured as years before the transition point when the cure rate begins to decline steeply. Therefore, the module may consider a range of progressing cancer possibilities (different years of early warning) and a no-cancer (not present or not progressing) possibility defined by the no-cancer predicted values. For each of these possibilities a probability distribution may be constructed that may be characterized by a mean and by variation, which may be characterized by standard deviations. There are two sources of variation that may be considered. First, trend variation may be caused by possibly random variation in test results. Second, biologic variation may be caused by differences among men or for a specific man over time.

The approaches described herein can be used as an alternative method for creating the long-term probabilities, as shown on FIG. 4. The long-term probabilities module is split into a personalized probability distributions module and probabilities module (400) and a Bayes long-term probabilities module (401). The Bayes calculations in the second module (402) are discussed in the above incorporated references. The first module (400) is discussed below. The outputs of module 400 are probabilities of the observed trend results: PSA, PSAV, fPSA % and fPSAV % conditional on no cancer and cancer for various years (X). These are created using personal information and input from biologic and trend models, as disclosed below.

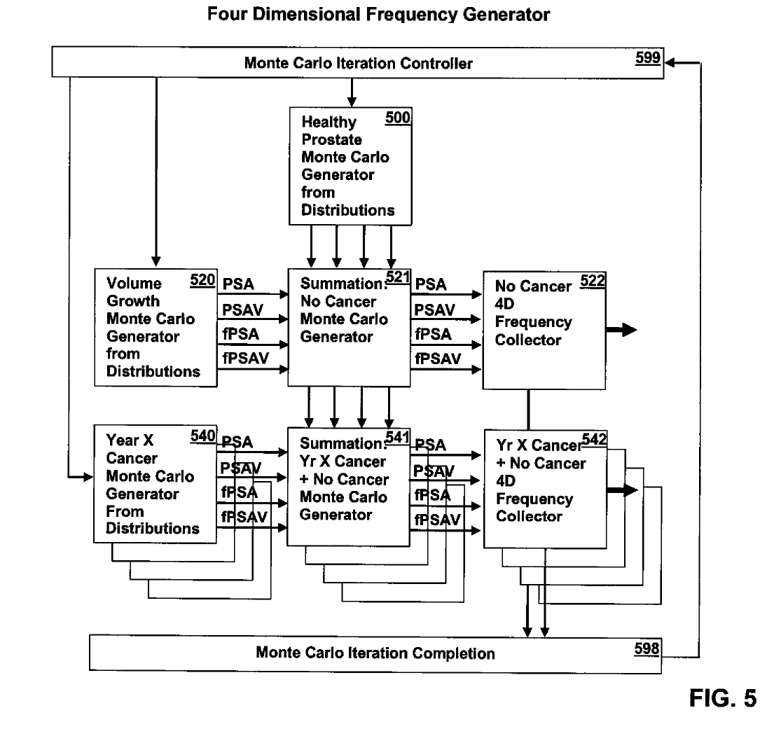

In an example embodiment, the personalized probability distributions and probabilities module uses a four dimensional frequency generator, shown on FIG. 5, which calculates personalized probability distributions and probabilities for cancer and no cancer cases in iterative fashion. Each iteration is initiated by the Monte Carlo iteration controller (599) and ended by the Monte Carlo iteration completion module (598), which returns control to the controller (599). For each iteration, trend values for a healthy prostate are generated from probability distributions in module 500. Trend values for prostate volume growth are generated from probability distributions in module 520. No cancer values are calculated in module 521 as the sum of values from module 500 and module 520. In module 522 the values for each iteration are added to the appropriate four dimensional bucket defined by ranges in four dimensions. As the number of iterations increase, frequency distributions for the no cancer case are built up in module 522 and output at the end of the process. For each iteration, trend values for each year X cancer case are generated from probability distributions in module 540. A range of cases are calculated for year X cancers, where X is a measure of cancer progression. Values for each year X cancer plus no cancer case are calculated in module 541 as the sum of values from module 540 and module 521. In module 542 the values for each iteration are added to the appropriate four dimensional bucket defined by ranges in four dimensions. As the number of iterations increase, frequency distributions for each year X cancer plus no cancer case are built up in module 542 and output at the end of the process.

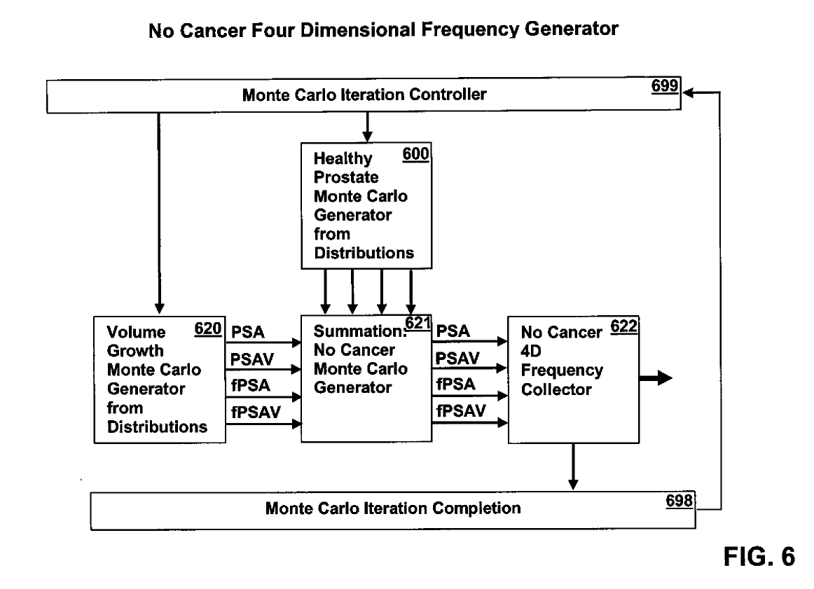

At times, it is computationally more efficient to use independent Monte Carlo processes for the no cancer case and cancer plus no cancer cases. An example four dimensional frequency generator, shown on FIG. 6, calculates personalized probability distributions and probabilities for the no cancer case in iterative fashion. Each iteration is initiated by the Monte Carlo iteration controller (699) and ended by the Monte Carlo iteration completion module (698), which returns control to the controller (699). For each iteration, trend values for a healthy prostate are generated from probability distributions in module 600. Trend values for prostate volume growth are generated from probability distributions in module 620. No cancer values are calculated in module 621 as the sum of values from module 600 and module 620. In module 622 the values for each iteration are added to the appropriate four dimensional bucket defined by ranges in four dimensions. As the number of iterations increase, frequency distributions for the no cancer case are built up in module 622.

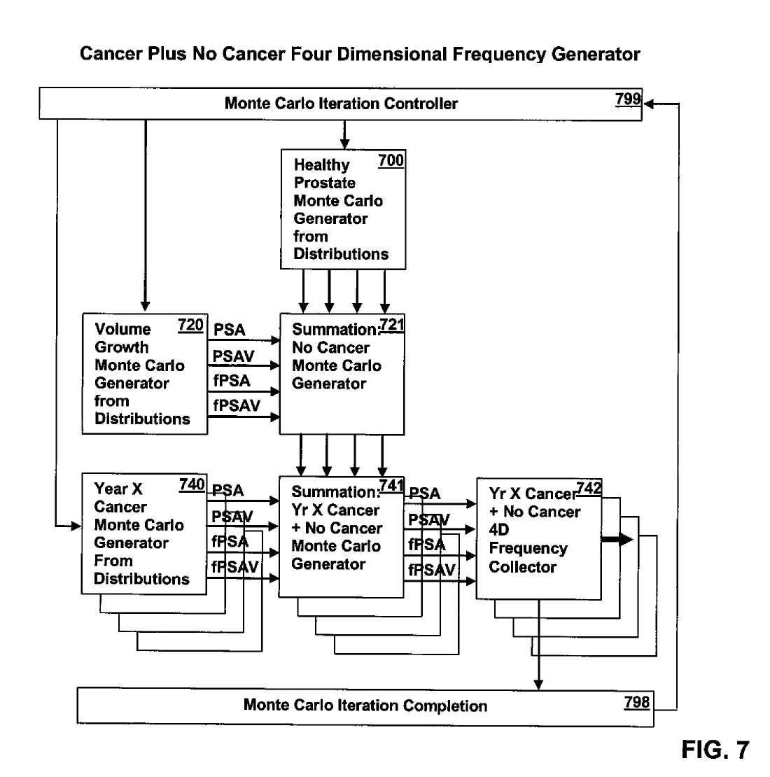

An example four dimensional frequency generator, shown on FIG. 7, calculates personalized probability distributions and probabilities for cancer plus no cancer cases in iterative fashion. Each iteration is initiated by the Monte Carlo iteration controller (799) and ended by the Monte Carlo iteration completion module (798), which returns control to the controller (799). For each iteration, trend values for a healthy prostate are generated from probability distributions in module 700. Trend values for prostate volume growth are generated from probability distributions in module 720. No cancer values are calculated in module 721 as the sum of values from module 700 and module 720. For each iteration, trend values for each year X cancer case are generated from probability distributions in module 740. A range of cases are calculated for year X cancers, where X is a measure of cancer progression. Values for each year X cancer plus no cancer case are calculated in module 741 as the sum of values from module 740 and module 721. In module 742 the values for each iteration are added to the appropriate four dimensional bucket defined by ranges in four dimensions. As the number of iterations increase, frequency distributions for each year X cancer plus no cancer case are built up in module 742.

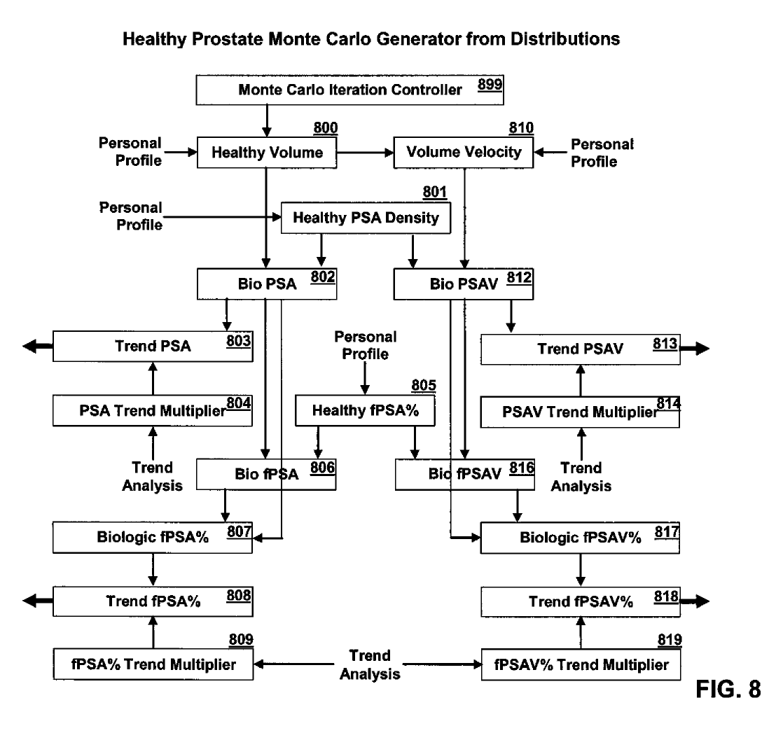

FIG. 8 shows an example Monte Carlo process in modules 500, 600 and 700 for generating outcomes for a healthy prostate from a number of probability distributions. Each iteration is initiated by the Monte Carlo iteration controller (899). A volume for a healthy prostate is drawn from a probability distribution in module 800. The nature and values of the distribution are affected by data from the personal profile. For example, age may influence the distribution and a volume measurement will strongly influence the distribution. Volume velocity for a healthy prostate is drawn from a probability distribution in module 810. The nature and values of the distribution are affected by the volume drawn in module 800 and data from the personal profile. For example, the mean and standard deviation for the volume velocity distribution tend to be larger for larger volumes. PSA density for a healthy prostate is drawn from a probability distribution in module 801. The nature and values of the distribution are affected by data from the personal profile. For example, age may influence the distribution; and past PSA and volume measurements may strongly influence the distribution if they are available. The flow chart implicitly assumes that the PSA density of new healthy prostate tissue has the same PSA density as old healthy prostate tissue. If future research indicates they are different then a second healthy PSAV density module would be used with number 811. A value for biologic PSA is calculated in module 802 as the product of the healthy PSA density from module 801 and the volume of a healthy prostate from module 800. In a similar way, a value for biologic PSAV is calculated in module 812 as the product of the healthy PSA density from module 801 and the volume velocity of a healthy prostate from module 810. Trend PSA (803) is the PSA trend multiplier (804) multiplied by biologic PSA (802), and trend PSAV (813) is the PSAV trend multiplier (814) multiplied by biologic PSAV (812). Trend variables add trend variation to biologic outcomes in order to simulate uncertainty in observed trend results. Trend multipliers (804 and 814) typically have a mean of 1.0 and standard deviations equal to the coefficients of variation (CV) for each of the estimated trends (where CV=SD/Mean). The CVs are obtained from analysis of PSA trends. A value for biologic free PSA is calculated in module 806 as the product of the healthy free PSA % from module 805 and biologic PSA from module 802. In a similar way, a value for biologic free PSAV is calculated in module 816 as the product of the healthy free PSA % from module 805 and biologic PSAV from module 812. The free PSA % for a healthy prostate is drawn from a probability distribution in module 805. The nature and values of the distribution are affected by data from the personal profile. For example, age may influence the distribution; and past PSA and free PSA results will strongly influence the distribution if they are available. The flow chart implicitly assumes that the free PSA % of new healthy prostate tissue has the same free PSA % as old healthy prostate tissue. If future research indicates they are different then a second healthy free PSAV % module would be used with number 815. A value for biologic free PSA % is calculated in module 807 as healthy biologic free PSA from module 806 divided by biologic PSA from module 802. In a similar way, a value for biologic free PSAV % is calculated in module 817 as healthy free PSAV from module 816 divided by biologic PSAV from module 812. Trend free PSA % (808) is the free PSA % trend multiplier (809) multiplied by biologic free PSA % (807), and trend free PSAV % (818) is the free PSAV % trend multiplier (819) multiplied by biologic free PSAV % (817). Trend variables add trend variation to biologic outcomes in order to simulate observed trend results. Trend multipliers (809 and 819) typically have a mean of 1.0 and standard deviations equal to the coefficients of variation (CV) for each of the estimated trends (where CV=SD/Mean). The CVs are obtained from analysis of free PSA and PSA trends. There are four outputs of this module, shown by the thick black arrows: trend PSA (803), trend PSAV (813), trend free PSA % (808) and trend free PSAV % (818).

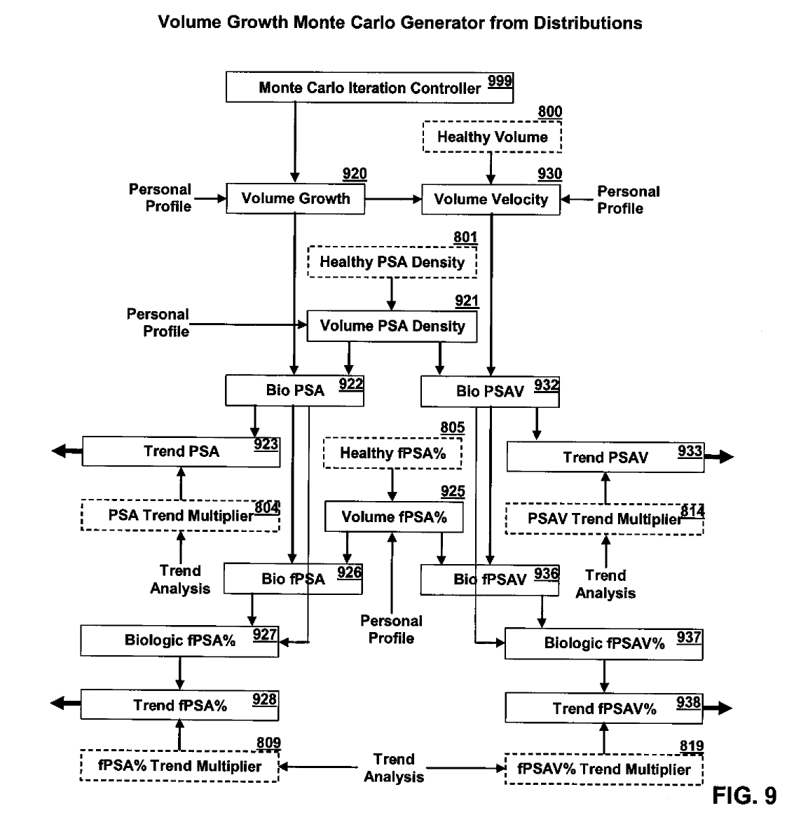

FIG. 9 shows an example Monte Carlo process in modules 500, 600 and 700 for generating outcomes for prostate volume growth from a number of probability distributions. Each iteration is initiated by the Monte Carlo iteration controller (999). A volume for volume growth is drawn from a probability distribution in module 920. The nature and values of the distribution are affected by data from the personal profile. For example, age may influence the distribution and a volume measurement will strongly influence the distribution. Volume velocity for volume growth is drawn from a probability distribution in module 930. The nature and values of the distribution are affected by the healthy volume drawn in module 800, the volume for volume growth drawn in module 920 and data from the personal profile. For example, the mean and standard deviation for the volume velocity distribution tend to be larger for larger volumes. PSA density for volume growth is drawn from a probability distribution in module 921. The nature and values of the distribution are affected by healthy PSA density (801) and data from the personal profile. For example, age may influence the distribution; and past PSA and volume measurements may strongly influence the distribution if they are available. The flow chart implicitly assumes that the PSA density of new volume growth has the same PSA density as old volume growth. If future research indicates they are different then a second volume growth PSAV density module would be used with number 931. A value for biologic PSA is calculated in module 922 as the product of the volume growth PSA density from module 921 and the volume of volume growth from module 920. In a similar way, a value for biologic PSAV is calculated in module 932 as the product of volume growth PSA density from module 921 and the volume velocity for volume growth from module 930. Trend PSA (923) is the PSA trend multiplier (804) multiplied by biologic PSA (922), and trend PSAV (933) is the PSAV trend multiplier (814) multiplied by biologic PSAV (932). A value for biologic free PSA is calculated in module 926 as the product of the volume growth free PSA % from module 925 and biologic PSA from module 922. In a similar way, a value for biologic free PSAV is calculated in module 936 as the product of the volume growth free PSA % from module 925 and biologic PSAV from module 922. The free PSA % for volume growth is drawn from a probability distribution in module 925. The nature and values of the distribution are affected by the healthy free PSA % (805) and data from the personal profile. For example, age may influence the distribution; and past PSA and free PSA results will strongly influence the distribution if they are available. The flow chart implicitly assumes that the free PSA % for volume growth has the same free PSA % as old volume growth. If future research indicates they are different then a second volume growth free PSAV % module would be used with number 935. A value for biologic free PSA % is calculated in module 927 as volume growth biologic free PSA from module 926 divided by biologic PSA from module 922. In a similar way, a value for biologic free PSAV % is calculated in module 936 as volume growth free PSAV from module 936 divided by biologic PSAV from module 932. Trend free PSA % (928) is the free PSA % trend multiplier (809) multiplied by biologic free PSA % (927), and trend free PSAV % (938) is the free PSAV % trend multiplier (819) multiplied by biologic free PSAV % (937). There are four outputs of this module, shown by the thick black arrows: trend PSA (923), trend PSAV (933), trend free PSA % (928) and trend free PSAV % (938).

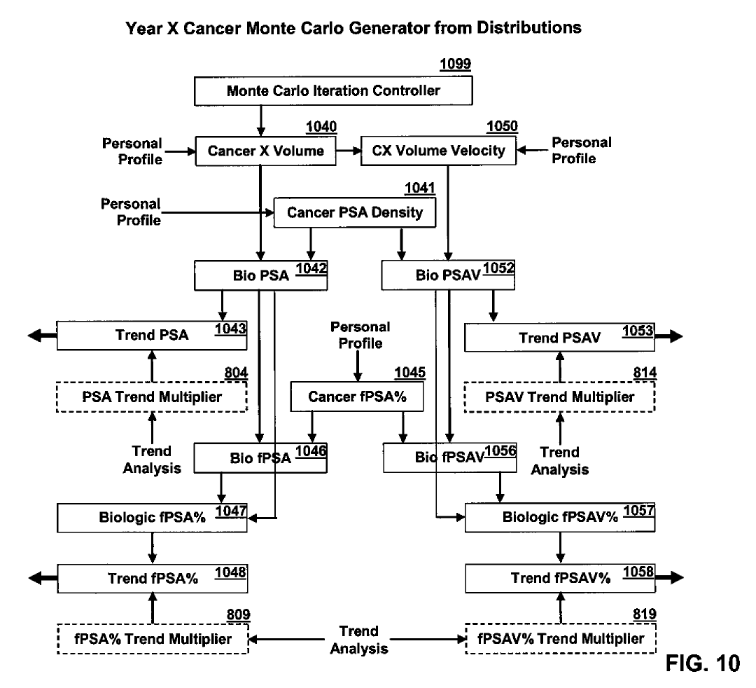

FIG. 10 shows an example Monte Carlo process in modules 500, 600 and 700 for generating outcomes for year X cancer from a number of probability distributions, where X is a measure of cancer progression. For example, X can be measured as the number of years before or after the Transition Point, defined as the time of progression when the cure rate begins to decline steeply. Other reference points for measuring X may work as well. In this example, fifteen year X cases might be considered: 2 and 1 years after the transition point, 0 years (at the transition point), and 1-12 years before the transition point. In this example, there can be fifteen parallel versions of FIG. 10, one for each year X case. Each iteration is initiated by the Monte Carlo iteration controller (1099). A volume for year X cancer is drawn from a probability distribution in module 1040. The nature and values of the distribution may be affected by data from the personal profile. For example, age may influence the distribution. Volume velocity for year X cancer is drawn from a probability distribution in module 1050. The nature and values of the distribution are affected by the volume for cancer X drawn in module 1040 and data from the personal profile. For example, the mean and standard deviation for the volume velocity distribution tend to be larger for larger volumes. PSA density for year X cancer is drawn from a probability distribution in module 1041. The nature and values of the distribution may be affected by data from the personal profile. For example, age may influence the distribution; and past PSA and volume measurements may strongly influence the distribution if they are available. The flow chart implicitly assumes that the PSA density of new year X cancer has the same PSA density as old year X cancer. If future research indicates they are different then a second year X cancer PSAV density module would be used with number 1051. A value for biologic PSA is calculated in module 1042 as the product of the year X cancer PSA density from module 1041 and the volume of year X cancer from module 1040. In a similar way, a value for biologic PSAV is calculated in module 1052 as the product of year X cancer PSA density from module 1041 and the volume velocity for year X cancer from module 1050. Trend PSA (1043) is the PSA trend multiplier (804) multiplied by biologic PSA (1042), and trend PSAV (1053) is the PSAV trend multiplier (814) multiplied by biologic PSAV (1052). Trend variables add trend variation to biologic outcomes in order to simulate observed trend results. Trend multipliers (804 and 814) typically have a mean of 1.0 and standard deviations equal to the coefficients of variation (CV) for each of the estimated trends (where CV=SD/Mean). The CVs are obtained from analysis of PSA trends. A value for biologic free PSA is calculated in module 1046 as the product of the year X cancer free PSA % from module 1045 and biologic PSA from module 1042. In a similar way, a value for biologic free PSAV is calculated in module 1056 as the product of the year X cancer free PSA % from module 1045 and biologic PSAV from module 1052. The free PSA % for year X cancer is drawn from a probability distribution in module 1045. The nature and values of the distribution may be affected by data from the personal profile. For example, age may influence the distribution; and past PSA and free PSA results may strongly influence the distribution if they are available. The flow chart implicitly assumes that the free PSA % for year X cancer has the same free PSA % as old year X cancer. If future research indicates they are different then a second year X cancer free PSAV % module would be used with number 1055. A value for biologic free PSA % is calculated in module 1047 as year X cancer biologic free PSA from module 1046 divided by biologic PSA from module 1042. In a similar way, a value for biologic free PSAV % is calculated in module 1057 as year X cancer free PSAV from module 1056 divided by biologic PSAV from module 1052. Trend free PSA % (1048) is the free PSA % trend multiplier (809) multiplied by biologic free PSA % (1047), and trend free PSAV % (1058) is the free PSAV % trend multiplier (819) multiplied by biologic free PSAV % (1057). Trend variables add trend variation to biologic outcomes in order to simulate observed trend results. Trend multipliers (809 and 819) typically have a mean of 1.0 and standard deviations equal to the coefficients of variation (CV) for each of the estimated trends (where CV=SD/Mean). The CVs are obtained from analysis of free PSA and PSA trends. There are four outputs of this module, shown by the thick black arrows: trend PSA (1043), trend PSAV (1053), trend free PSA % (1048) and trend free PSAV % (1058).

The approach described in this example generates extensive four dimensional distributions that can be used to find the probabilities needed for the Bayes calculations of the probability of progressing cancer. However, the calculations can be time consuming and cause delays in real time responses to users. The approach of focused probabilities is discussed below to address this if it is an issue for a situation at hand. The number of calculations and the time to perform them can be reduced substantially by focusing narrowly on the probabilities needed for the Bayes calculations rather than on generating extensive four dimensional distributions. Additional discussion of methods for focusing on the needed probabilities is provided below.

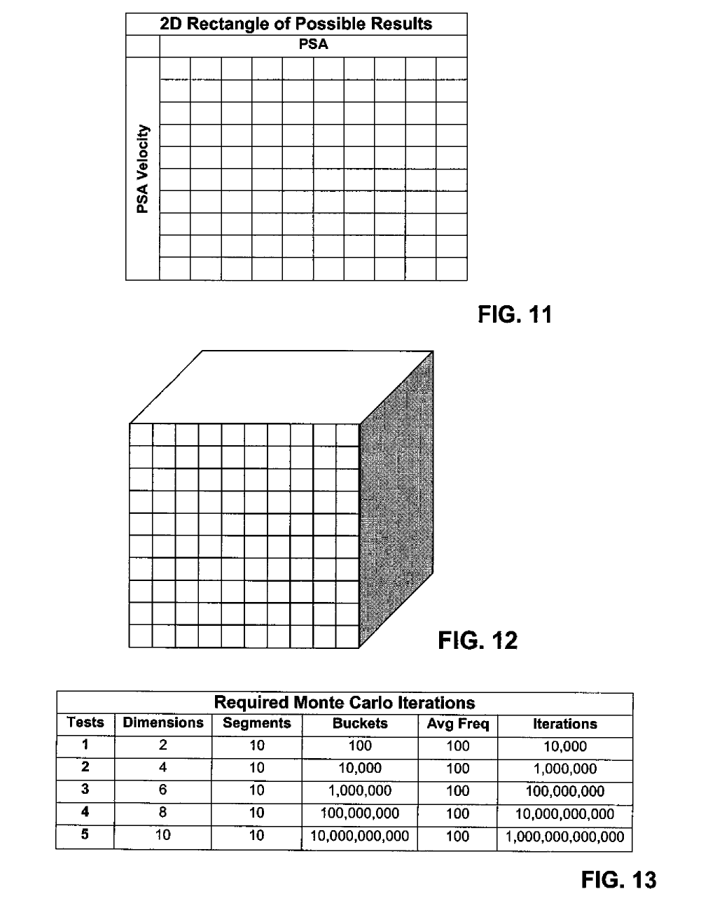

In an example for one biomarker, such as PSA, there is interest in two dimensions: PSA and PSA velocity (PSAV). A two dimensional rectangle of possible Monte Carlo results can be created by dividing each dimension into segments. FIG. 11 shows the 100 possible buckets of possible results when each dimension is divided into ten segments. The segments for each of the two dimensions might be: ten segments for the PSA dimension (for example, >=0 and <1; >=1 and <2; >=2 and <3; >=3 and <4; >=4 and <5; >=5 and <6; >=6 and <7; >=7 and <8; >=8 and <9; and >=9) and ten segments for the PSAV dimension (for example, >=0.0 and <0.1; >=0.1 and <0.2; >=0.2 and <0.3; >=0.3 and <0.4; >=0.4 and <0.5; >−0.5 and <0.6; >=0.6 and <0.7; >=0.7 and <0.8; >=0.8 and <0.9; and >=0.9).

For two tests, such as PSA and free PSA, there can be interest in four dimensions: PSA, PSAV, fPSA % and fPSAV %. A four dimensional hyper cube of possible Monte Carlo results can be created by dividing each dimension into segments. FIG. 12 suggests the 10,000 possible buckets of possible results when each dimension is divided into ten segments (even though it can depict only three of the four dimensions).

An example number of Monte Carlo iterations required to create a reasonably stable distribution increases exponentially with the number of tests, as shown by the table of FIG. 13. The first column shows the number of biomarker tests considered. Some drug testing companies that a panel of tests will become standard screening practice in the future for conditions such as prostate cancer. The second column shows the corresponding number of dimensions when both trend values and velocities are considered for each test. The third column shows the ten segments assumed for each dimension. The fourth column shows the corresponding number of buckets for which frequencies are collected to create the overall multidimensional probability distributions. The fifth column shows the 100 average frequency collected in each bucket. An average frequency of this magnitude is needed to assure at least a few results collected in the buckets on the low frequency tails of the distributions.

The table of FIG. 13 suggests that a trillion Monte Carlo iterations would be required for each case if a panel of five screening tests become the standard for a condition such as prostate cancer. This creates an enormous problem for an on-line service the offers real time analysis. The delays would be unacceptable using the fastest computers available now or in the near future.

Example Monte Carlo calculations for one personalized case requires the frequency for only one bucket rather than the frequencies for all possible buckets. Consider a man concerned about prostate cancer with a series of PSA biomarker results from blood tests, Trends can be estimated for each biomarker and analyzed using methods previously disclosed. The results might be: trend PSA (3.0±0.4) and trend PSA velocity (0.40±0.20). The bucket used to collect the frequency of this outcome might be: (PSA 3.0±0.5) or (PSA>2.5 and <3.5) and (PSAV 0.4±0.05) or (PSAV>0.35 and <0.45).

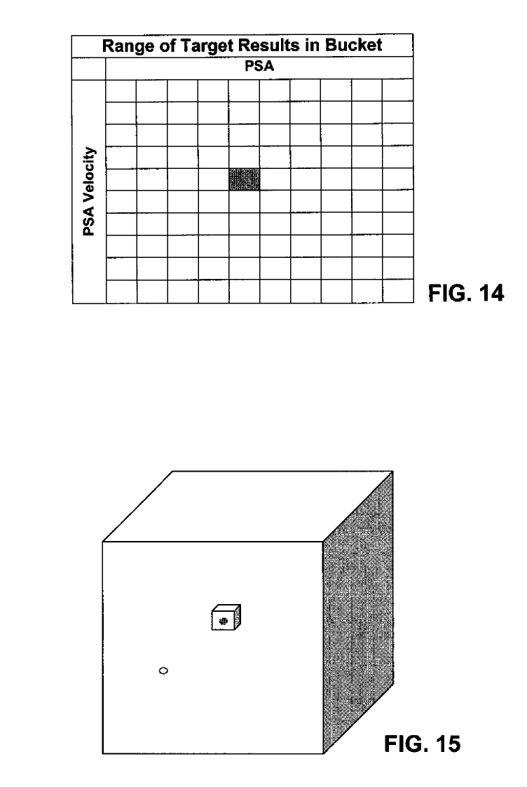

The gray rectangle on the table of FIG. 14 shows conceptually the bucket of concern defined by the range of PSA and PSAV results around the observed trend results. For one example, there is no interested in any of the other buckets that are not shaded.

In yet another example, consider a man concerned about prostate cancer with a series of PSA and free PSA biomarker results from blood tests. Trends can be estimated for each biomarker and analyzed using methods previously disclosed. The results might be: trend PSA (3.0±0.4), trend PSA velocity (0.40±0.20), trend free PSA % (17.0%±2.0%), and trend free PSA velocity % (6.0%±3.0%). The bucket used to collect the frequency of this outcome might be: (a) (PSA=3.0±0.5) or (PSA>2.5 and <3.5) and (b) (PSAV=0.4±0.05) or (PSAV>0.35 and <0.45) and (c) (fPSA % 17.0%±2.0%) or (fPSA %>15.0% and <19.0%), and (d) (fPSAV % 6.0%±2.0%) or (fPSAV %>4.0% and <8.0%).

The small cube inside the large cube shown by FIG. 15 suggests conceptually the hypercube bucket of concern defined by the range of PSA, PSAV, fPSA % and fPSAV % results around the observed trend results (even though it can depict only three of the four dimensions). For one case, there is no interested in any of the other buckets that are outside the small cube. In an example for a single case, trend values are known for PSA, PSAV, fPSA % and fPSAV %, which is a point in the 4D hyper cube. A small hyper cube bucket can be created around the point to collect Monte Carlo results that fall within the ranges. The frequency of the results in the bucket can be used to estimate the probability of the results. Monte Carlo results that fall with the bucket, like the solid dot in the small cube of FIG. 15, are recorded; and results that fall outside the bucket, like the circle in the large cube of FIG. 15, are not recorded.

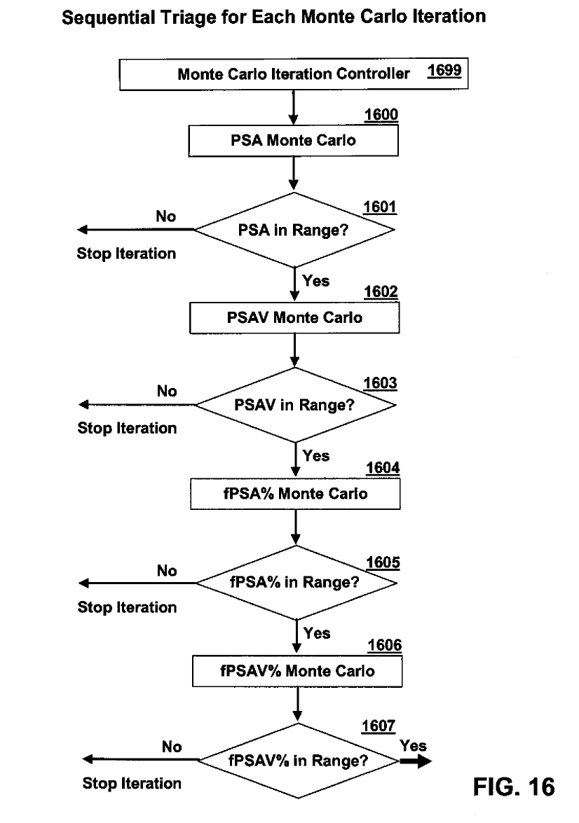

Example methods for reducing the number of calculations by focusing on the bucket of concern are disclosed below. They are elaborations of the approach shown on FIG. 16. The Monte Carlo processes disclosed above are reorganized to calculate one dimension at a time, in this case: PSA in module 1600, PSAV in module 1602, fPSA % in module 1604, and fPSAV % in module 1606. For each iteration controlled by module 1699, PSA is calculated in module 1600 using Monte Carlo methods. As controlled by module 1601, the process stops for this iteration if PSA falls outside of the target range of the bucket, but the process continues if PSA falls within the target range of the bucket. If the iteration continues, PSAV is calculated in module 1602 using Monte Carlo methods. As controlled by module 1603, the process stops for this iteration if PSAV falls outside of the target range of the bucket, but the process continues if PSAV falls within the target range of the bucket. If the iteration continues, fPSA % is calculated in module 1604 using Monte Carlo methods. As controlled by module 1605, the process stops for this iteration if fPSA % falls outside of the target range of the bucket, but the process continues if fPSA % falls within the target range of the bucket. If the iteration continues, fPSAV % is calculated in module 1606 using Monte Carlo methods. As controlled by module 1607, the process stops for this iteration if fPSAV % falls outside of the target range of the bucket, but the process continues if fPSAV % falls within the target range of the bucket. The term sequential triage is used to describe stopping the iteration at each stage as soon as it is known that the result will miss the target bucket. Many unnecessary calculations can be avoided using this method.

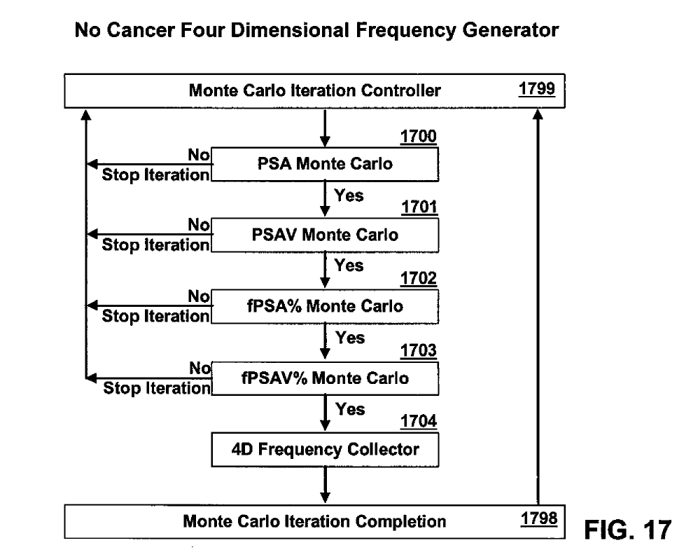

FIG. 17 shows an example four dimensional frequency generator for the no cancer case. Each iteration is initiated by the Monte Carlo iteration controller (1799). For each iteration, PSA is calculated in module 1700 using Monte Carlo methods. The process stops for this iteration if PSA falls outside of the target range of the bucket, but the process continues if PSA falls within the target range of the bucket. If the iteration continues, PSAV is calculated in module 1701 using Monte Carlo methods. The process stops for this iteration if PSAV falls outside of the target range of the bucket, but the process continues if PSAV falls within the target range of the bucket. If the iteration continues, fPSA % is calculated in module 1702 using Monte Carlo methods. The process stops for this iteration if fPSA % falls outside of the target range of the bucket, but the process continues if fPSA % falls within the target range of the bucket. If the iteration continues, fPSAV % is calculated in module 1703 using Monte Carlo methods. The process stops for this iteration if fPSAV % falls outside of the target range of the bucket, but the process continues if fPSAV % falls within the target range of the bucket. The four dimensional frequency collector (1704) keeps track of the number of Monte Carlo iterations started and the number of outcomes that fall in the 4D bucket. Frequency is calculated by dividing the number of outcomes in the bucket by the number of iterations started. Finally, control is passed to the Monte Carlo iteration completion module (1798).

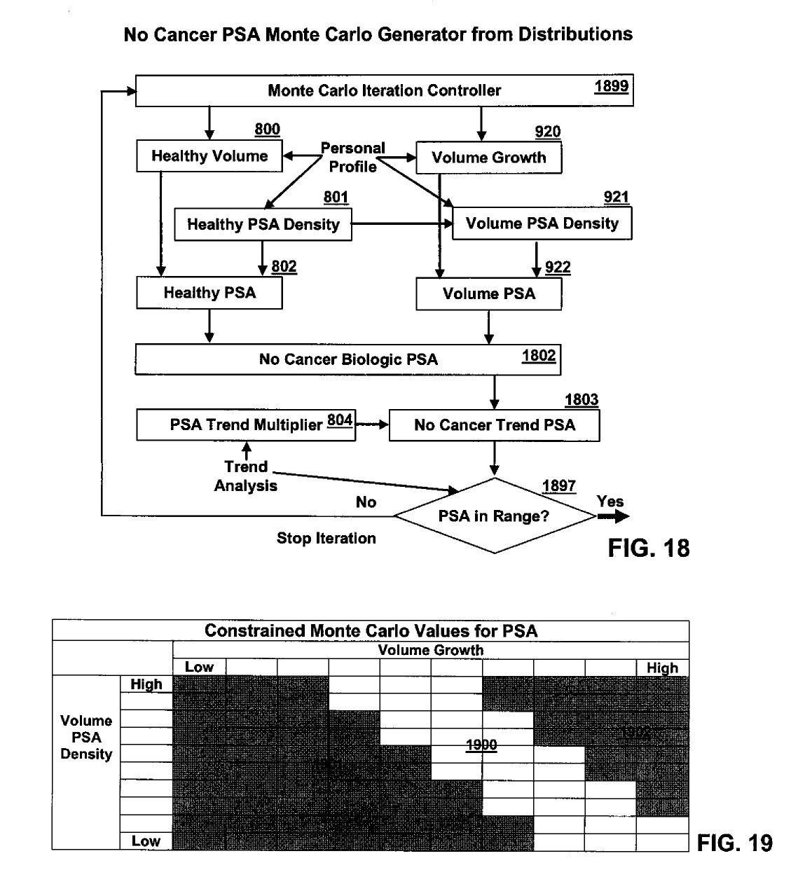

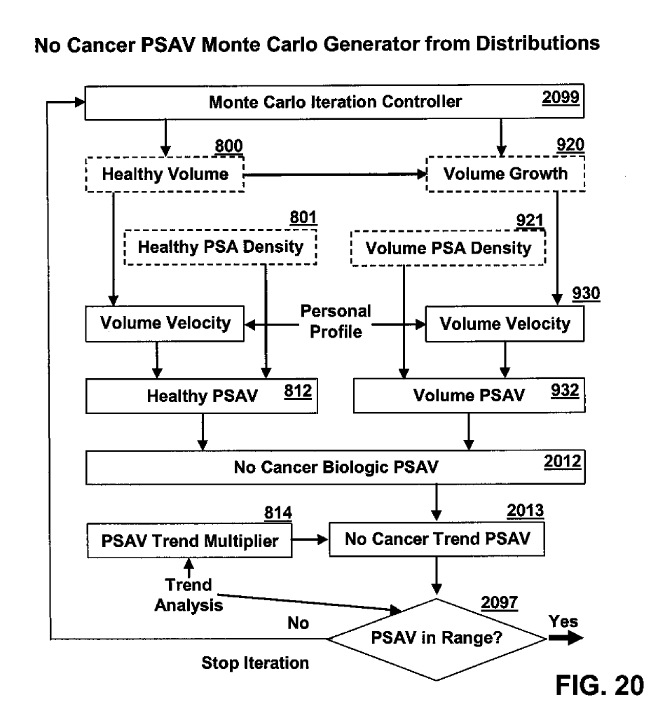

FIG. 18 shows an example Monte Carlo process for generating no cancer PSA outcomes from a number of probability distributions. Each iteration is initiated by the Monte Carlo iteration controller (1899). A volume for a healthy prostate is drawn from a probability distribution in module 800. The nature and values of the distribution are affected by data from the personal profile. For example, age may influence the distribution and a volume measurement will strongly influence the distribution. PSA density for a healthy prostate is drawn from a probability distribution in module 801. The nature and values of the distribution are affected by data from the personal profile. For example, age may influence the distribution; and past PSA and volume measurements may strongly influence the distribution if they are available. A value for healthy biologic PSA is calculated in module 802 as the product of the healthy PSA density from module 801 and the volume of a healthy prostate from module 800. A volume for volume growth is drawn from a probability distribution in module 920. The nature and values of the distribution are affected by data from the personal profile. For example, age may influence the distribution and a volume measurement will strongly influence the distribution. PSA density for volume growth is drawn from a probability distribution in module 921. The nature and values of the distribution are affected by healthy PSA density (801) and data from the personal profile. For example, age may influence the distribution; and past PSA and volume measurements may strongly influence the distribution if they are available. A value for volume growth biologic PSA is calculated in module 922 as the product of the volume growth PSA density from module 921 and the volume of volume growth from module 920. No cancer biologic PSA is calculated in module 1802 as the sum of healthy biologic PSA from module 802 and volume growth biologic PSA from module 922. No cancer trend PSA (1803) is the previously drawn PSA trend multiplier (804) multiplied by no cancer biologic PSA (1802). Trend multipliers (804) typically have a mean of 1.0 and standard deviations equal to the coefficients of variation (CV) for each of the estimated trends (where CV=SD/Mean). The CVs are obtained from analysis of PSA trends. Module 1897 evaluates whether the no cancer trend PSA from module 1803 is within the target range. If the decision is yes to continue the iteration for year X cancer the system may then proceed to module 2099 on FIG. 20 to start the process for no cancer PSAV. Trend PSA from module 1897 is the one output of this module, shown by the thick black arrow.

As suggested by FIG. 19, total calculation time can be reduced by constraining the range of values used to calculate PSA to the combinations of values (the not shaded area 1900) that are likely to result in trend PSA values that are within range of the target value checked in module 1897. Healthy PSA falls within a relatively narrow range with high probability. Therefore, most of the variation in no cancer PSA is caused by volume growth. For a given target PSA range in module 1897, there are combinations of low volume growth and/or low volume PSA density shown by shaded area 1901 that are very likely to result in no cancer trend PSA values (1803) that are less than the target range. These combinations of values need not used in the Monte Carlo process. In a similar way for a given target PSA range in module 1897, there are combinations of high volume growth and/or high volume PSA density shown by shaded area 1902 that are very likely to result in no cancer trend PSA values (1803) that are greater than the target range. These combinations of values need not used in the Monte Carlo process. Only combinations of values in the not shaded area 1900 needed to be considered in the Monte Carlo process. However, the overall number of iterations considered needs to include the number of iterations that would have been generated for the shaded areas 1901 and 1902, as well as for the not shaded area 1900.

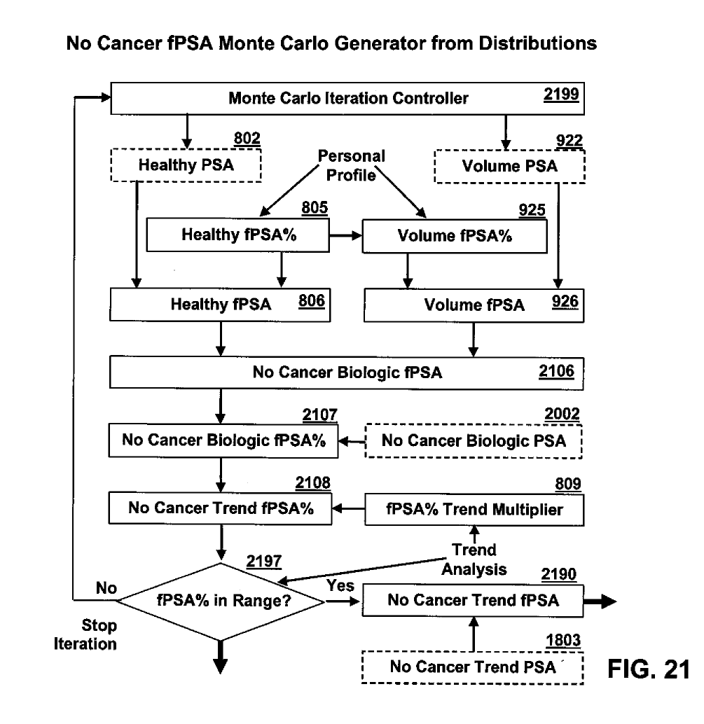

FIG. 20 shows an example Monte Carlo process for generating no cancer PSAV outcomes from a number of probability distributions. Each iteration is initiated by the Monte Carlo iteration controller (2099). Volume velocity for a healthy prostate is drawn from a probability distribution in module 810. The nature and values of the distribution are affected by the volume previously drawn in module 800 and data from the personal profile. For example, the mean and standard deviation for the volume velocity distribution tend to be larger for larger volumes. PSA density for a healthy prostate was previously drawn from a probability distribution in module 801. The flow chart implicitly assumes that the PSA density of new healthy prostate tissue has the same PSA density as old healthy prostate tissue. If future research indicates they are different then a second healthy PSAV density module would be used with number 811. A value for healthy biologic PSAV is calculated in module 812 as the product of the healthy PSA density from module 801 and the volume velocity of a healthy prostate from module 810. Volume velocity for volume growth is drawn from a probability distribution in module 930. The nature and values of the distribution are affected by the healthy volume drawn in module 800, the volume for volume growth drawn in module 920 and data from the personal profile. For example, the mean and standard deviation for the volume velocity distribution tend to be larger for larger volumes. PSA density for volume growth was previously drawn from a probability distribution in module 921. The flow chart implicitly assumes that the PSA density of new volume growth has the same PSA density as old volume growth. If future research indicates they are different then a second volume growth PSAV density module would be used with number 931. A value for volume growth biologic PSAV is calculated in module 932 as the product of volume growth PSA density from module 921 and the volume velocity for volume growth from module 930. No cancer biologic PSAV is calculated in module 2012 as the sum of healthy biologic PSAV from module 812 and volume growth biologic PSA from module 932. No cancer trend PSAV (2013) is the previously drawn PSAV trend multiplier (814) multiplied by no cancer biologic PSAV (2012). Trend multipliers (814) typically have a mean of 1.0 and standard deviations equal to the coefficients of variation (CV) for each of the estimated trends (where CV=SD/Mean). The CVs are obtained from analysis of PSA trends. Module 2097 evaluates whether the no cancer trend PSAV from module 2013 is within the target range. If the decision is yes to continue the iteration for year X cancer the system may then proceed to module 2199 on FIG. 21 to start the process for no cancer free PSA. Trend PSAV from module 2097 is the one output of this module, shown by the thick black arrow.

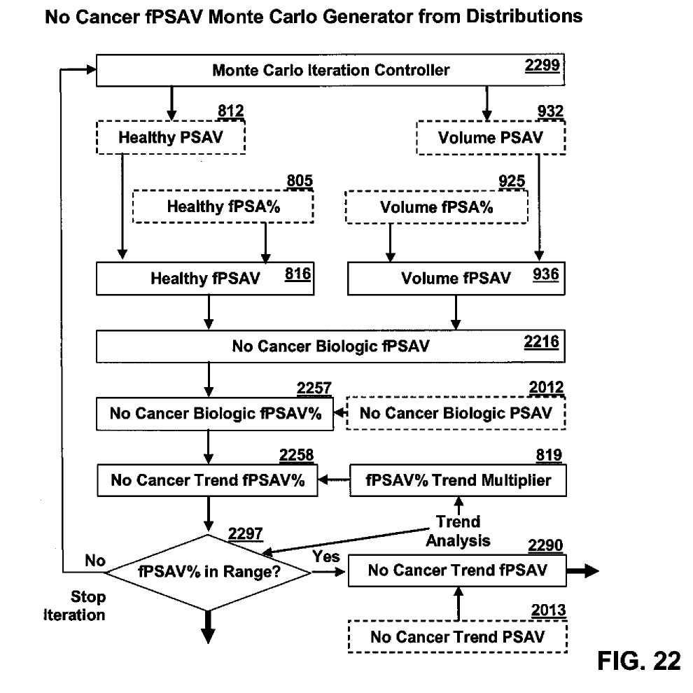

FIG. 21 demonstrates an example Monte Carlo process for generating no cancer free PSA outcomes from a number of probability distributions. Each iteration is initiated by the Monte Carlo iteration controller (2199). A value for healthy biologic free PSA is calculated in module 806 as the product of the healthy free PSA % from module 805 and biologic PSA from module 802. The free PSA % for a healthy prostate is drawn from a probability distribution in module 805. The nature and values of the distribution are affected by data from the personal profile. For example, age may influence the distribution; and past PSA and free PSA results will strongly influence the distribution if they are available. A value for volume growth biologic free PSA is calculated in module 926 as the product of the volume growth free PSA % from module 925 and biologic PSA from module 922. The free PSA % for volume growth is drawn from a probability distribution in module 925. The nature and values of the distribution are affected by the healthy free PSA % (805) and data from the personal profile. For example, age may influence the distribution; and past PSA and free PSA results will strongly influence the distribution if they are available. No cancer biologic free PSA is calculated in module 2106 as the sum of healthy biologic fPSA from module 806 and volume growth biologic fPSA from module 926. A value for no cancer biologic free PSA % is calculated in module 2107 as biologic free PSA from module 2106 divided by biologic PSA from module 2002. Trend free PSA % (2108) is the free PSA % trend multiplier (809) multiplied by biologic free PSA % (2107). Trend variables add trend variation to biologic outcomes in order to simulate observed trend results. Trend multipliers (809) typically have a mean of 1.0 and standard deviations equal to the coefficients of variation (CV) for each of the estimated trends (where CV=SD/Mean). The CVs are obtained from analysis of free PSA and PSA trends. Module 2197 evaluates whether the no cancer trend fPSA % from module 2108 is within the target range. If the decision is yes to continue the iteration the system may calculate trend fPSA in module 2190 and then proceed to module 2299 on FIG. 22 to start the process for no cancer free PSAV. Trend fPSA is calculated in module 2190 as trend fPSA % (2197) multiplied by trend PSA (1803). Trend fPSA % (2197) and trend fPSA (2190) are the two outputs of this module, shown by the thick black arrows.

FIG. 22 shows an example Monte Carlo process for generating free PSAV outcomes from a number of probability distributions. Each iteration is initiated by the Monte Carlo iteration controller (2299). A value for healthy biologic free PSAV is calculated in module 816 as the product of the healthy free PSA % previously drawn from module 805 and previously calculated biologic PSAV from module 812. The flow chart implicitly assumes that the free PSA % of new healthy prostate tissue has the same free PSA % as old healthy prostate tissue. If future research indicates they are different then a second healthy free PSAV % module would be used with number 815 instead of module 805. A value for volume growth biologic free PSAV is calculated in module 936 as the product of the volume growth free PSA % previously drawn from module 925 and previously calculated biologic PSAV from module 932. The flow chart implicitly assumes that the free PSA % for volume growth has the same free PSA % as old volume growth. If future research indicates they are different then a second volume growth free PSAV % module would be used with number 935 instead of module 925. No cancer biologic free PSAV is calculated in module 2216 as the sum of healthy biologic fPSAV from module 816 and volume growth biologic fPSAV from module 936. A value for no cancer biologic free PSAV % is calculated in module 2257 as biologic free PSAV from module 2216 divided by previously calculated biologic PSAV from module 2012. Trend free PSA V % (2258) is the free PSAV % trend multiplier (819) multiplied by biologic free PSAV % (2257). Trend variables add trend variation to biologic outcomes in order to simulate observed trend results. Trend multipliers (819) typically have a mean of 1.0 and standard deviations equal to the coefficients of variation (CV) for each of the estimated trends (where CV=SD/Mean). The CVs are obtained from analysis of free PSA and PSA trends. Module 2297 evaluates whether the no cancer trend fPSAV % from module 2258 is within the target range. If the decision is yes to continue the iteration the system may calculate trend fPSAV in module 2290 and then return control to the iteration controller. Trend fPSAV is calculated in module 2290 as trend fPSAV % (2297) multiplied by previously calculated trend PSAV (2013). Trend fPSA % (2297) and trend fPSA (2290) are the two value outputs of this module, shown by the thick black arrows. Methods for efficiently calculating year X cancer probabilities needed for the Bayes calculations are disclosed below.

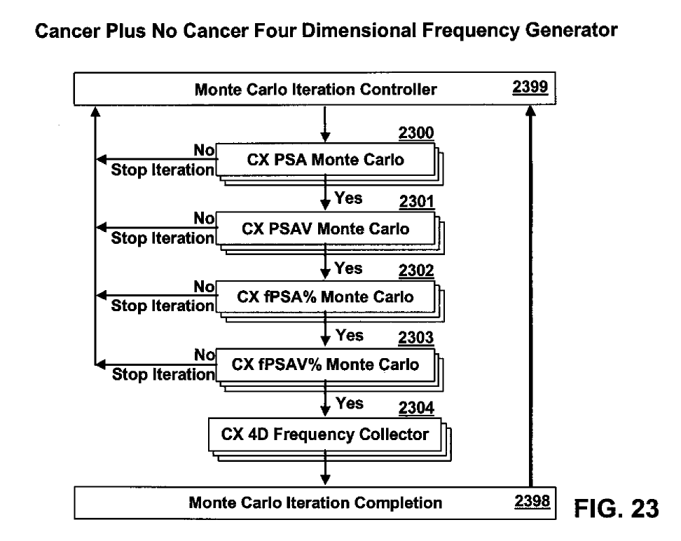

FIG. 23 shows an example four dimensional frequency generator for each year X cancer plus no cancer case. Each iteration is initiated by the Monte Carlo iteration controller (2399). For each iteration, PSA is calculated in module 2300 using Monte Carlo methods. The process stops for this iteration if PSA falls outside of the target range of the bucket, but the process continues if PSA falls within the target range of the bucket. If the iteration continues, PSAV is calculated in module 2301 using Monte Carlo methods. The process stops for this iteration if PSAV falls outside of the target range of the bucket, but the process continues if PSAV falls within the target range of the bucket. If the iteration continues, fPSA % is calculated in module 2302 using Monte Carlo methods. The process stops for this iteration if fPSA % falls outside of the target range of the bucket, but the process continues if fPSA % falls within the target range of the bucket. If the iteration continues, fPSAV % is calculated in module 2303 using Monte Carlo methods. The process stops for this iteration if fPSAV % falls outside of the target range of the bucket, but the process continues if fPSAV % falls within the target range of the bucket. The four dimensional frequency collector (2304) keeps track of the number of Monte Carlo iterations started and the number of outcomes that fall in the 4D bucket. Frequency is calculated by dividing the number of outcomes in the bucket by the number of iterations started. Finally, control is returned to the Monte Carlo iteration completion module (2398).

FIG. 23 shows an example Monte Carlo process for generating outcomes for year X cancer from a number of probability distributions, where X is a measure of cancer progression. For example, X can be measured as the number of years before or after the Transition Point, defined as the time of progression when the cure rate begins to decline steeply. Other reference points for measuring X may work as well. In this example, fifteen year X cases might be considered: 2 and 1 years after the transition point, 0 years (at the transition point), and 1-12 years before the transition point. In this example, there will be fifteen parallel versions of FIG. 10, one for each year X case.

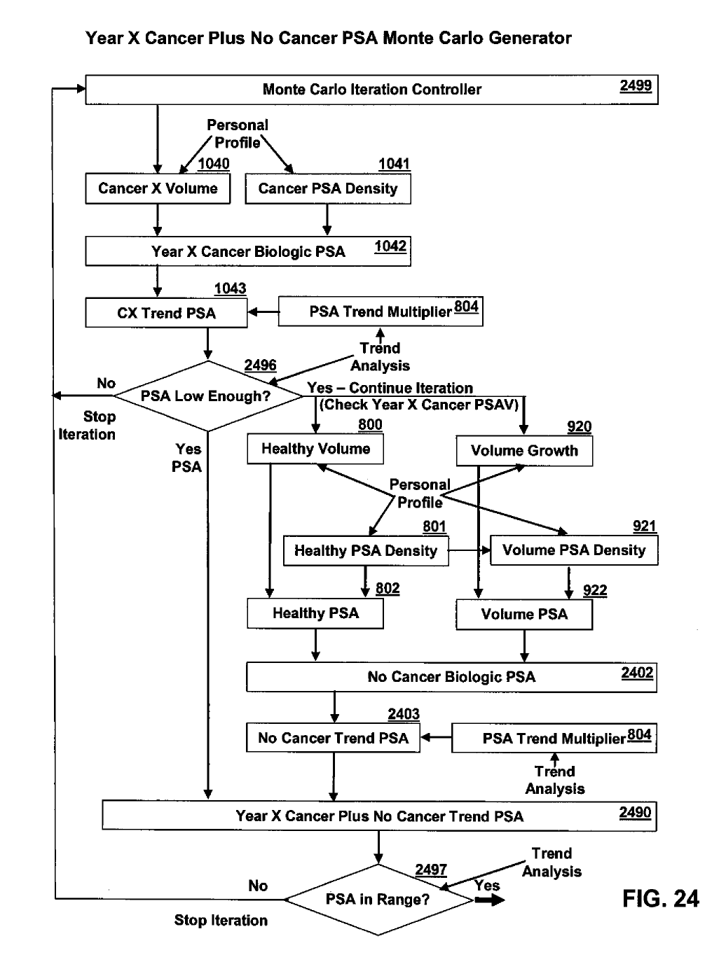

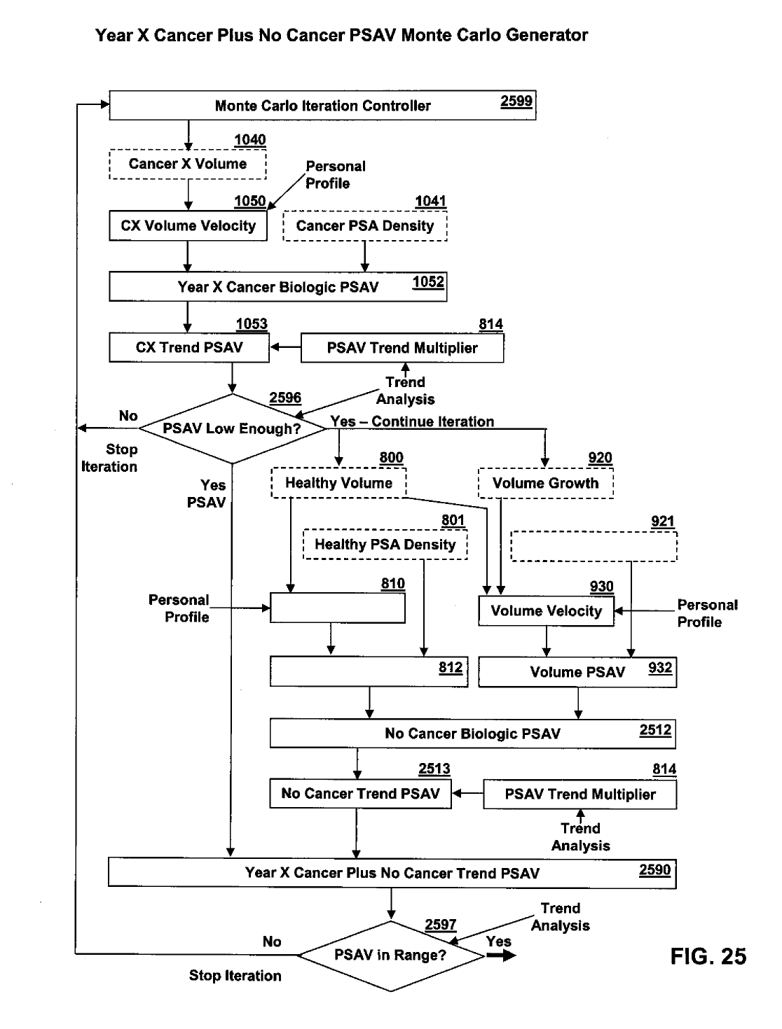

FIG. 24 shows an example Monte Carlo process for generating year X cancer plus no cancer PSA outcomes from a number of probability distributions. Each iteration is initiated by the Monte Carlo iteration controller (2499). A volume for year X cancer is drawn from a probability distribution in module 1040. The nature and values of the distribution may be affected by data from the personal profile. For example, age may influence the distribution. A value for biologic PSA is calculated in module 1042 as the product of the year X cancer PSA density from module 1041 and the volume of year X cancer from module 1040. Trend PSA (1043) is the PSA trend multiplier (804) multiplied by biologic PSA (1042), trend variables add trend variation to biologic outcomes in order to simulate observed trend results. Trend multipliers (804) typically have a mean of 1.0 and standard deviations equal to the coefficients of variation (CV) for each of the estimated trends (where CV=SD/Mean). The CVs are obtained from analysis of PSA trends. Module 2496 evaluates whether each year X trend PSA from module 1043 is low enough compared to the observed trend PSA to warrant continuation of the iteration. If a year X trend (1043) exceeds the target range then it is clear the combined trend PSA (2490) will exceed the target range and the iteration should be stopped for the year X cancer case. More complex decision rules may be used to eliminate iterations that have a low probability of meeting the final test in module 2497 and avoid unnecessary calculations. If the decision is yes to continue the iteration for year X cancer the system may then proceed to module 2590 on FIG. 25 to check and see if year X trend PSAV from module 2553 is low enough compared to the observed trend PSAV to warrant continuation of the iteration. A volume for a healthy prostate is drawn from a probability distribution in module 800. The nature and values of the distribution are affected by data from the personal profile. For example, age may influence the distribution and a volume measurement will strongly influence the distribution. PSA density for a healthy prostate is drawn from a probability distribution in module 801. The nature and values of the distribution are affected by data from the personal profile. For example, age may influence the distribution; and past PSA and volume measurements may strongly influence the distribution if they are available. A value for biologic PSA is calculated in module 802 as the product of the healthy PSA density from module 801 and the volume of a healthy prostate from module 800. A volume for volume growth is drawn from a probability distribution in module 920. The nature and values of the distribution are affected by data from the personal profile. For example, age may influence the distribution and a volume measurement will strongly influence the distribution. PSA density for volume growth is drawn from a probability distribution in module 921. The nature and values of the distribution are affected by healthy PSA density (801) and data from the personal profile. For example, age may influence the distribution; and past PSA and volume measurements may strongly influence the distribution if they are available. A value for biologic PSA is calculated in module 922 as the product of the volume growth PSA density from module 921 and the volume of volume growth from module 920. No cancer biologic PSA is calculated in module 2402 as the sum of healthy biologic PSA from module 802 and volume growth biologic PSA from module 922. No cancer trend PSA (2403) is the previously drawn PSA trend multiplier (804) multiplied by no cancer biologic PSA (2402). Year X cancer plus no cancer trend PSA is calculated in module 2490 as the sum of year X cancer trend PSA from module 2496 and no cancer trend PSA from module 2403. Module 2497 evaluates whether each year X cancer plus no cancer trend PSA from module 2490 is within the target range. If the decision is yes to continue the iteration for year X cancer the system may then proceed to module 2599 on FIG. 25 to start the process for year X trend PSAV. Trend PSA (2497) is the one output of this module, shown by the thick black arrow.

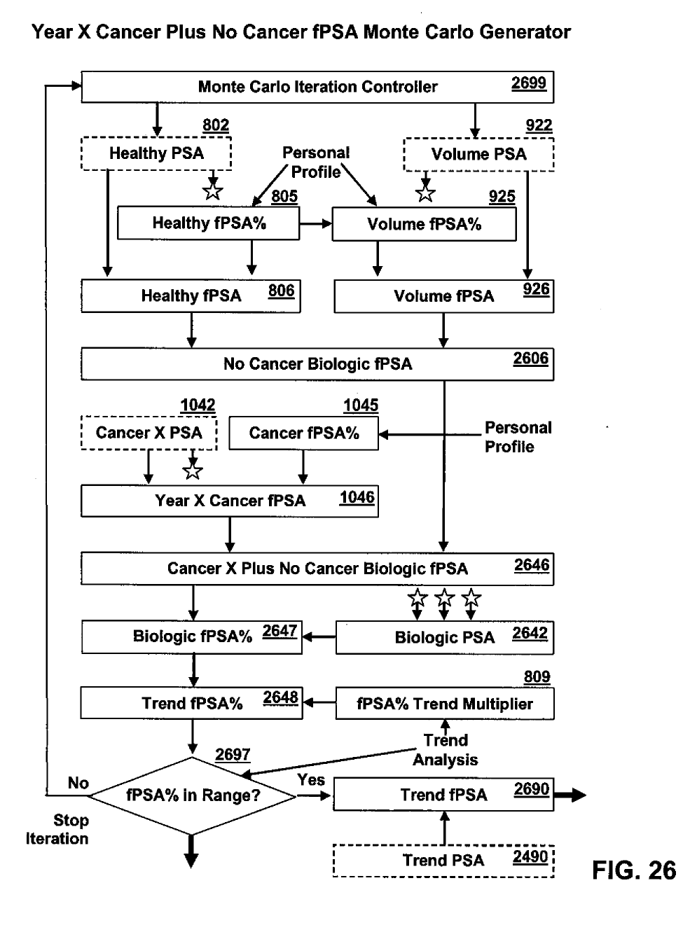

FIG. 25 demonstrates an Monte Carlo process for generating year X cancer plus no cancer PSAV outcomes from a number of probability distributions. Each iteration is initiated by the Monte Carlo iteration controller (2599). A volume velocity for year X cancer is drawn from a probability distribution in module 1050. The nature and values of the distribution are affected by the volume for cancer X drawn in module 1040 and data from the personal profile. For example, the mean and standard deviation for the volume velocity distribution tend to be larger for larger volumes. A value for biologic PSAV is calculated in module 1052 as the product of the year X cancer PSA density from module 1041 and the volume velocity of year X cancer from module 1050. Trend PSAV (1053) is the PSAV trend multiplier (814) multiplied by biologic PSAV (1052), trend variables add trend variation to biologic outcomes in order to simulate observed trend results. Trend multipliers (814) typically have a mean of 1.0 and standard deviations equal to the coefficients of variation (CV) for each of the estimated trends (where CV=SD/Mean). The CVs are obtained from analysis of PSA trends. Module 2596 evaluates whether each year X trend PSAV from module 1053 is low enough compared to the observed trend PSAV to warrant continuation of the iteration. If a year X trend (1053) exceeds the target range then it is clear the combined trend PSA (2590) will exceed the target range and the iteration should be stopped for the year X cancer case. More complex decision rules may be used to eliminate iterations that have a low probability of meeting the final test in module 2597 and avoid unnecessary calculations. Volume velocity for a healthy prostate is drawn from a probability distribution in module 810. The nature and values of the distribution are affected by the volume previously drawn in module 800 and data from the personal profile. For example, the mean and standard deviation for the volume velocity distribution tend to be larger for larger volumes. PSA density for a healthy prostate was previously drawn from a probability distribution in module 801. The flow chart implicitly assumes that the PSA density of new healthy prostate tissue has the same PSA density as old healthy prostate tissue. If future research indicates they are different then a second healthy PSAV density module would be used with number 811. A value for biologic PSAV is calculated in module 812 as the product of the healthy PSA density from module 801 and the volume velocity of a healthy prostate from module 810. Volume velocity for volume growth is drawn from a probability distribution in module 930. The nature and values of the distribution are affected by the healthy volume drawn in module 800, the volume for volume growth drawn in module 920 and data from the personal profile. For example, the mean and standard deviation for the volume velocity distribution tend to be larger for larger volumes. PSA density for volume growth was previously drawn from a probability distribution in module 921. The flow chart implicitly assumes that the PSA density of new volume growth has the same PSA density as old volume growth. If future research indicates they are different then a second volume growth PSAV density module would be used with number 931. A value for biologic PSAV is calculated in module 932 as the product of volume growth PSA density from module 921 and the volume velocity for volume growth from module 930. No cancer biologic PSAV is calculated in module 2512 as the sum of healthy biologic PSAV from module 812 and volume growth biologic PSA from module 932. No cancer trend PSAV (2513) is the previously drawn PSAV trend multiplier (814) multiplied by no cancer biologic PSAV (2512). Year X cancer plus no cancer trend PSAV is calculated in module 2590 as the sum of year X cancer trend PSAV from module 2596 and no cancer trend PSAV from module 2513. Module 2597 evaluates whether each year X cancer plus no cancer trend PSAV from module 2590 is within the target range. If the decision is yes to continue the iteration for year X cancer the system may then proceed to module 2699 on FIG. 26 to start the process for year X trend free PSA. Trend PSAV (2597) is the one value output of this module, shown by the thick black arrow.

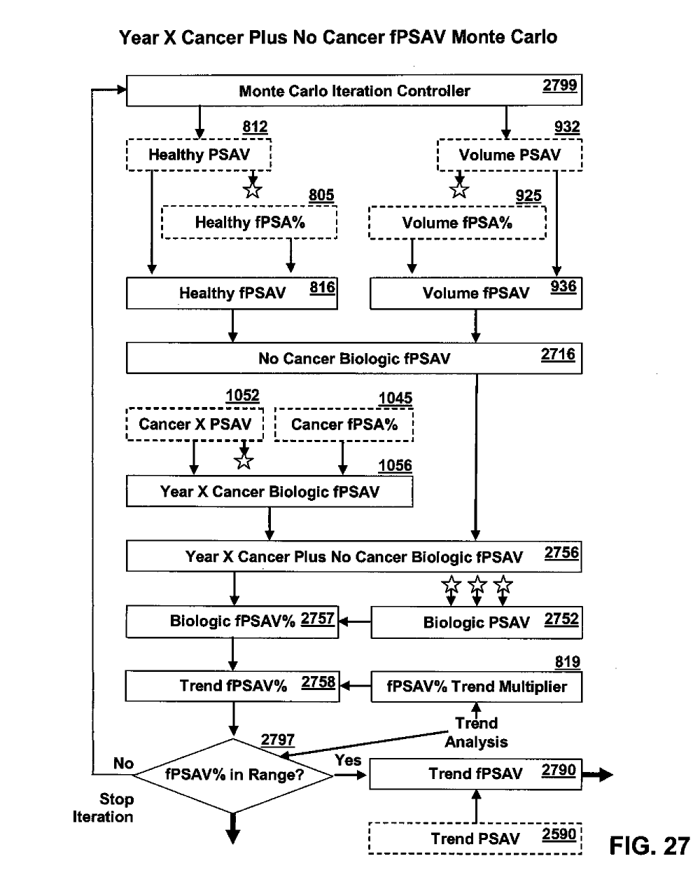

FIG. 26 shows an example Monte Carlo process for generating year X cancer plus no cancer free PSA outcomes from a number of probability distributions. Each iteration is initiated by the Monte Carlo iteration controller (2699). A value for healthy biologic free PSA is calculated in module 806 as the product of the healthy free PSA % from module 805 and biologic PSA from module 802. The free PSA % for a healthy prostate is drawn from a probability distribution in module 805. The nature and values of the distribution are affected by data from the personal profile. For example, age may influence the distribution; and past PSA and free PSA results will strongly influence the distribution if they are available. A value for volume growth biologic free PSA is calculated in module 926 as the product of the volume growth free PSA % from module 925 and biologic PSA from module 922. The free PSA % for volume growth is drawn from a probability distribution in module 925. The nature and values of the distribution are affected by the healthy free PSA % (805) and data from the personal profile. For example, age may influence the distribution; and past PSA and free PSA results will strongly influence the distribution if they are available. No cancer biologic free PSA is calculated in module 2606 as the sum of healthy biologic fSAV from module 806 and volume growth biologic PSA from module 926. A value for year X cancer biologic free PSA is calculated in module 1046 as the product of the year X cancer free PSA % from module 1045 and biologic PSA from module 1042. The free PSA % for year X cancer is drawn from a probability distribution in module 1045. The nature and values of the distribution may be affected by data from the personal profile. For example, age may influence the distribution; and past PSA and free PSA results may strongly influence the distribution if they are available. Year X cancer plus no cancer biologic fPSA is calculated in module 2646 as the sum of year X cancer fPSA from module 1046 and no cancer fPSA from module 2606. A value for year X cancer plus no cancer biologic free PSA % is calculated in module 2647 as biologic free PSA from module 2646 divided by biologic PSA from module 2642. Biologic PSA in module 2642 is calculated as the sum of healthy, volume growth and year X cancer biologic PSAs from modules 802, 922 and 1042 respectively. Trend free PSA % (2648) is the free PSA % trend multiplier (809) multiplied by biologic free PSA % (2647). Trend variables add trend variation to biologic outcomes in order to simulate observed trend results. Trend multipliers (809) typically have a mean of 1.0 and standard deviations equal to the coefficients of variation (CV) for each of the estimated trends (where CV=SD/Mean). The CVs are obtained from analysis of free PSA and PSA trends. Module 2697 evaluates whether each year X cancer plus no cancer trend fPSA % from module 2648 is within the target range. If the decision is yes to continue the iteration for year X cancer the system may calculate trend fPSA in module 2690 and then proceed to module 2799 on FIG. 27 to start the process for year X trend free PSAV. Trend fPSA is calculated in module 2690 as trend fPSA % (2697) multiplied by previously calculated trend PSA (2490). Trend fPSA % (2697) and trend fPSA (2690) are the two outputs of this module, shown by the thick black arrows.

FIG. 27 shows an example Monte Carlo process for generating year X cancer plus no cancer free PSAV outcomes from a number of probability distributions. Each iteration is initiated by the Monte Carlo iteration controller (2799). A value for healthy biologic free PSAV is calculated in module 816 as the product of the healthy free PSA % previously drawn from module 805 and biologic PSAV from module 812. The flow chart implicitly assumes that the free PSA % of new healthy prostate tissue has the same free PSA % as old healthy prostate tissue. If future research indicates they are different then a second healthy free PSAV % module would be used with number 815 instead of module 805. A value for volume growth biologic free PSAV is calculated in module 936 as the product of the volume growth free PSA % previously drawn from module 925 and biologic PSAV from module 932. The flow chart implicitly assumes that the free PSA % for volume growth has the same free PSA % as old volume growth. If future research indicates they are different then a second volume growth free PSAV % module would be used with number 935 instead of module 925. No cancer biologic free PSAV is calculated in module 2716 as the sum of healthy biologic fPSAV from module 816 and volume growth biologic fPSAV from module 936. A value for year X cancer biologic free PSAV is calculated in module 1056 as the product of the year X cancer free PSA % from module 1045 and biologic PSA from module 1052. The free PSA % for year X cancer was previously drawn from a probability distribution in module 1045. The flow chart implicitly assumes that the free PSA % for year X cancer has the same free PSA % as old year X cancer. If future research indicates they are different then a second year X cancer free PSAV % module would be used with number 1055 instead of module 1045. Year X cancer plus no cancer biologic fPSAV is calculated in module 2756 as the sum of year X cancer fPSAV from module 1056 and no cancer fPSAV from module 2616. A value for year X cancer plus no cancer biologic free PSAV % is calculated in module 2757 as biologic free PSAV from module 2756 divided by biologic PSAV from module 2752. Biologic PSAV in module 2752 is calculated as the sum of healthy, volume growth and year X cancer biologic PSAVs from modules 812, 932 and 1052 respectively. Trend free PSA V % (2758) is the free PSAV % trend multiplier (819) multiplied by biologic free PSAV % (2757). Trend variables add trend variation to biologic outcomes in order to simulate observed trend results. Trend multipliers (819) typically have a mean of 1.0 and standard deviations equal to the coefficients of variation (CV) for each of the estimated trends (where CV=SD/Mean). The CVs are obtained from analysis of free PSA and PSA trends. Module 2797 evaluates whether each year X cancer plus no cancer trend fPSAV % from module 2758 is within the target range. If the decision is yes to continue the iteration for year X cancer the system may calculate trend fPSAV in module 2790 and then return control to the iteration controller. Trend fPSAV is calculated in module 2790 as trend fPSAV % (2797) multiplied by previously calculated trend PSAV (2590). Trend fPSA % (2697) and trend fPSA (2690) are the two outputs of this module, shown by the thick black arrows.

Warnings and alerts may be triggered by variables in the dynamic screening analysis system and may determine choices of custom content. Warnings may be triggered when a combination of the probability of a medical condition and the years of early warning reach predetermined levels. Alerts may be triggered when a combination of residual velocities and strength of evidence reach predetermined levels.

A warning status may determine custom content in reports to users. Warning levels may be triggered when specified variables reach predetermined levels, either individually or in combination with other specified variables. Variables that may trigger cancer warnings include the probability of progressing cancer and the number of years of early warning.

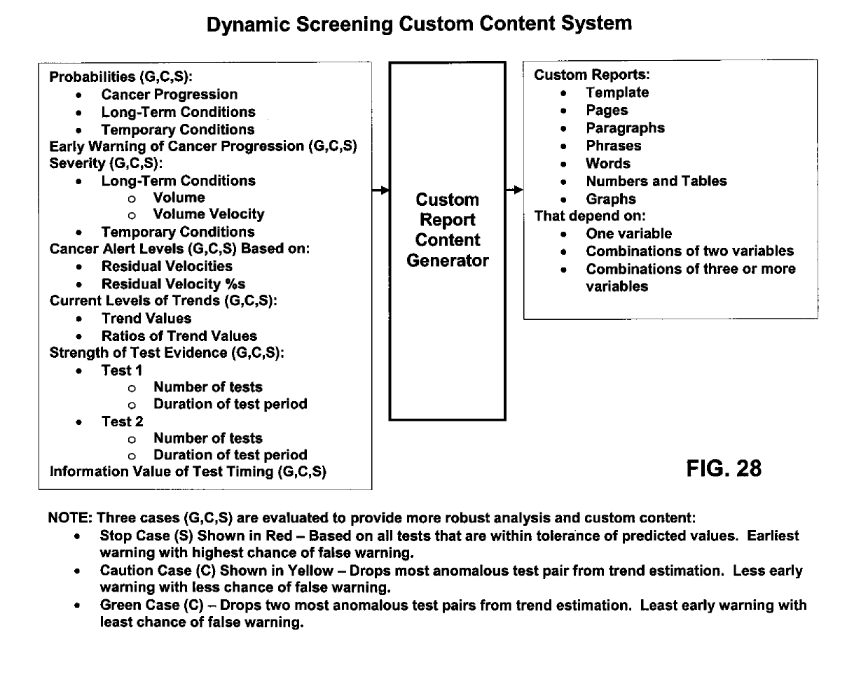

A high level block diagram of an example custom content system might function is shown in FIG. 28. Custom content includes words, paragraphs, numbers, tables, graphs and other content used in custom reports produced by the system and suggested by the list of outputs on the right of FIG. 28. Custom content can depend on one input variable or combinations of two or more variables suggested by the list of input variables on the left in FIG. 28. Custom content may take into account variations among the variables for the three cases: Red Stop, Yellow Caution and Green.

An example of custom content based on two variables is described below with brief custom content shown in italics below each combination of probability of progressing cancer and length of early warning of progressing cancer:
If Low probability of progressing cancer and Long early warning then content is: Wait patiently as continued testing decreases or increases the probability.
If High probability of progressing cancer and Long early warning then content is: Explore treatments and timing in a deliberate manner because the patient has time.
If Low probability of progressing cancer and Short early warning then content is: Test intensively because time is short in the unlikely event cancer is progressing.
If High probability of progressing cancer and Short early warning then content is: Schedule best treatment quickly because the patient is short of time.

Feedback can be a part of improving the accuracy and reliability of one or more of the disclosed systems and methods. Evaluation of the experience of many men using disclosed approaches can provide better estimates of the values and probabilities of many of the variables used in the analysis. The results of each individual evaluation are combined with others and analyzed as a group to create summaries of all screening histories.

It can be less difficult to evaluate individual experience looking backward than it is to predict it looking forward. For example, looking backward allows one to separate individuals into two groups: men who have experienced progressing cancer and men who have not. This knowledge removes an uncertainty from the analysis and allows precise estimation of the contributions of progressing cancer.

Improving the ability to predict outcomes and estimate the probability distributions of those outcomes is a central part of the feedback learning process. In an embodiment, multi-dimensional response surfaces can be developed where possible to fine tune the predictions and estimates based on a variety of variables that may include age, race and other demographic variables. Response surfaces can be estimated using standard statistical methods, such as multiple regression analysis. They can be used for two groups of men: men without progressing cancer; and men with progressing cancer.

The following are two examples of what one can expect to learn. For men without progressing cancer, the stability of the velocity densities can be a determinant of one's confidence in the predictions of PSA and free PSA. One may be able to learn more about how it behaves through feedback learning. For men with progressing cancer, the joint probability of concurrent changes in the residual free PSA velocity % and similar variables can improve the confidence in early warning.

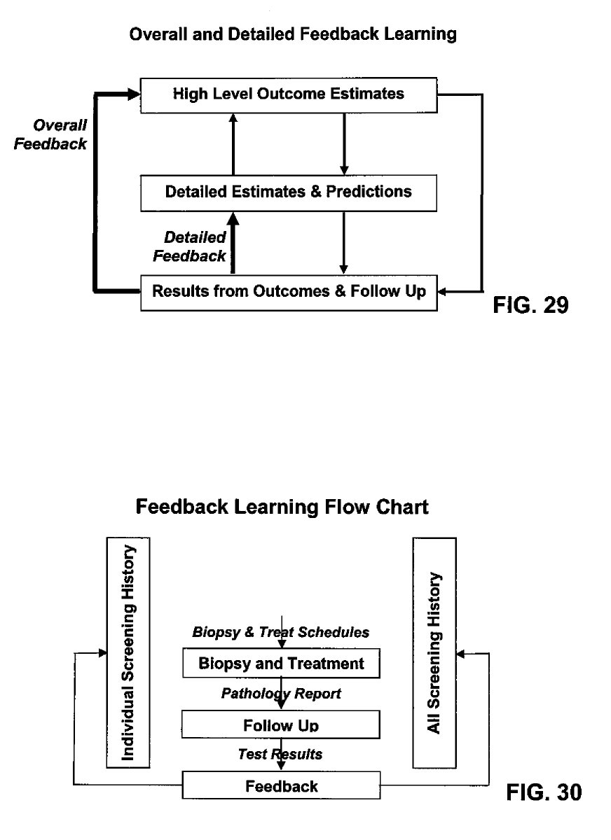

In an example, two types of feedback learning can improve the method over time, as suggested by the flow chart in FIG. 29. Detailed feedback can improve the accuracy of estimates and predictions. Overall feedback can allow one to make sure that estimates of high level outcomes based on detailed estimates and predictions can be unbiased and consistent with overall results. Two examples of high level outcomes are progression probability and cure ratio. Detailed feedback can be collected for every variable (or important variable) used in the estimation and prediction process. Best estimates and probability distributions can be calculated and used in the estimation and prediction parts of the method. For example, PSA and free PSA velocity density can be considered important variables used in the prediction process for progression probability, as noted earlier. The probability distributions for predictions depend on how much those variables are likely to vary from year to year for a given man. Less variation for a wide range of men means a tighter probability distribution around the predictions based on those variables. Overall feedback calibrates the method so that estimates of high level outcomes using detailed methods are consistent with actual high level outcomes for groups of the population. For example, the average estimated probability for the whole population based on detailed methods should be consistent with the overall probability for the whole population. In addition, this consistency should be maintained for smaller groups of the population.

In an embodiment, the feedback process depends on gathering information about outcomes, as suggested by FIG. 30. Information about outcomes can be fed back to individual screening history and to all screening history for analysis of groups of individuals. For a biopsy, a doctor uses a device to inject thin hollow needles into the prostate to extract tissue. A pathologist exams the tissue and provides a diagnosis of prostate cancer if it exists. Primary treatment is intended to cure prostate cancer. It includes surgery to remove the prostate and various types of radiation to kill the cancer. A pathology report after surgery can provide useful information about the progress of cancer. The results of these pathology reports can provide useful feedback about outcomes that can allow one to improve the effectiveness of the method. The follow up module is on FIG. 30. PSA tests and periodic physicals are used to follow patients' progress after treatment or no treatment depending on their choice. PSA tests are used to determine recurrence and the early progress of the disease. Later symptoms, metastasis and eventually death can be followed for many men. Feedback of these outcomes can help one improve the effectiveness of the method, as outlined in the next section. The feedback module is on FIG. 30. Decisions and results for each man can be analyzed to learn what actually happened. The results can be pooled with others and analyzed for common trends and probability distributions of outcomes. The distributions can be combined with information from a single man to improve predictions and estimates of probabilities, especially for progression.

In another aspect of the invention, a medical information system for assessing a disease of a subject is provided that comprises: an input device for receiving subject data corresponding to a biomarker for the disease at least two different times, wherein the data corresponding to the at least two different times form a first trend; a processor that assesses a probability of said trend relating to historical data; a storage unit in communication with the processor having a database for: (i) storing the subject data; (ii) storing historical data related to the disease; and an output device that transmits information relating to the probability of said trend relating to historical data to an end user.

The invention also provides a method for assessing a disease in a subject comprising: collecting data from the subject corresponding to a biomarker for the disease at least two different times, wherein the data corresponding to the at least two different times form a trend; exporting said data for manipulation of said data by executing a method of the invention; and importing the results of said manipulation to an end user. For example, data is collected at a first location, such as a hospital, the data is exported to a second location, such as a remote server in any remote location, where a method of the invention is executed to obtain information regarding the disease in a subject, and then the information is imported from the remote location back to the first location, such as the point-of-care in the hospital, or the information is imported to a third location, such as a database.

It is further noted that the systems and methods may be implemented on various types of computer architectures, such as for example on a networked system or in a client-server configuration, or in an application service provider configuration, on a single general purpose computer or workstation. The systems and methods may include data signals conveyed via networks (for example, local area network, wide area network, internet, combinations thereof), fiber optic medium, carrier waves, wireless networks. for communication with one or more data processing devices. The data signals can carry any or all of the data disclosed herein (for example, user input data, the results of the analysis to a user) that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein.

The systems' and methods' data (for example, associations, mappings) may be stored and implemented in one or more different types of computer-implemented ways, such as different types of storage devices and programming constructs (for example, data stores, RAM, ROM, Flash memory, flat files, databases, programming data structures, programming variables, IF-THEN (or similar type) statement constructs). It is noted that data structures describe formats for use in organizing and storing data in databases, programs, memory, or other computer-readable media for use by a computer program.

The systems and methods may be provided on many different types of computer-readable media including computer storage mechanisms (for example, CD-ROM, diskette, RAM, flash memory, computer's hard drive, magnetic tape, and holographic storage) that contain instructions (for example, software) for use in execution by a processor to perform the methods' operations and implement the systems described herein.

The computer components, software modules, functions, data stores and data structures described herein may be connected directly or indirectly to each other in order to allow the flow of data needed for their operations. It is also noted that the meaning of the term module includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

In general, in yet another aspect, a computer readable medium is provided including computer readable instructions, wherein the computer readable instructions instruct a processor to execute step a) of the methods described above. The instructions can operate in a software runtime environment.

In general, in yet another aspect, a data signal is provided that can be transmitted using a network, wherein the data signal includes said posterior probability calculated in step a) of the methods described above. The data signal can further include packetized data that is transmitted through wired or wireless networks.

In an aspect, a computer readable medium comprises computer readable instructions, wherein the instructions when executed carry out a calculation of the probability of a medical condition in a patient based upon data obtained from the patient corresponding to at least one biomarker. The computer readable instructions can operate in a software runtime environment of the processor. In an embodiment, a software runtime environment provides commonly used functions and facilities required by the software package. Examples of a software runtime environment include, but are not limited to, computer operating systems, virtual machines or distributed operating systems. As will be appreciated by those of ordinary skill in the art, several other examples of runtime environment exist. The computer readable instructions can be packaged and marketed as a software product or part of a software package. For example, the instructions can be packaged with an assay kit for PSA.

The computer readable medium may be a storage unit of the present invention as described herein. It is appreciated by those skilled in the art that computer readable medium can also be any available media that can be accessed by a server, a processor, or a computer. The computer readable medium can be incorporated as part of the computer-based system of the present invention, and can be employed for a computer-based assessment of a medical condition.

In an embodiment, the calculation of a probability can be carried out on a computer system. The computer system can comprise any or all of the following: a processor, a storage unit, software, firmware, a network communication device, a display, a data input, and a data output. A computer system can be a server. A server can be a central server that communicates over a network to a plurality of input devices and/or a plurality of output devices. A server can comprise at least one storage unit, such as a hard drive or any other device for storing information to be accessed by a processor or external device, wherein the storage unit can comprise one or more databases. In an embodiment, a database can store hundreds to millions of data points corresponding to a biomarker from hundreds to millions of subjects. A storage unit can also store historical data read from an external database or as input by a user. In an embodiment, a storage unit stores data received from an input device that is communicating or has communicated with the server. A storage unit can comprise a plurality of databases. In an embodiment, each of a plurality of databases corresponds to each of a plurality of biomarkers. In another embodiment, each of a plurality of databases corresponds to each of a plurality of possible medical conditions of a subject. An individual database can also comprise information for a plurality of possible medical conditions or a plurality of biomarkers or both. Further, a computer system can comprise multiple servers.

A processor can access data from a storage unit or from an input device to perform a calculation of an output from the data. A processor can execute software or computer readable instructions as provided by a user, or provided by the computer system or server. The processor may have a means for receiving patient data directly from an input device, a means of storing the subject data in a storage unit and a means for processing data. The processor may also include a means for receiving instructions from a user or a user interface. The processor may have memory, such as random access memory, as is well known in the art. In one embodiment, an output that is in communication with the processor is provided.

After performing a calculation, a processor can provide the output, such as from a calculation, back to, for example, the input device or storage unit, to another storage unit of the same or different computer system, or to an output device. Output from the processor can be displayed by data display. A data display can be a display screen (for example, a monitor or a screen on a digital device), a print-out, a data signal (for example, a packet), an alarm (for example, a flashing light or a sound), a graphical user interface (for example, a webpage), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In an embodiment, an output device is the same device as the input device. Example output devices include, but are not limited to, a telephone, a wireless telophone, a mobile phone, a PDA, a flash memory drive, a light source, a sound generator, a fax machine, a computer, a computer monitor, a printer, an iPOD, and a webpage. The user station may be in communication with a printer or a display monitor to output the information processed by the server.

A client-server, relational database architecture can be used in embodiments of the invention. A client server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A database can be developed for a medical condition in which relevant information is filtered or obtained over a communication network (for example, the internet) from one or more data sources, such as a public remote database, an internal remote database, and a local database. A public database can include online sources of free data for use by the general public, such as, for example, databases supplied by the U.S. Department of Health and Human Services. For example, an internal database can be a private internal database belonging to particular hospital, or a SMS (Shared Medical system) for providing data. A local database can comprise, for example, biomarker data relating to a medical condition. The local database may include data from a clinical trial. It may also include data such as blood test results, patient survey responses, or other items from patients in a hospital.

Subject data can be stored with a unique identifier for recognition by a processor or a user. In another step, the processor or user can conduct a search of stored data by selecting at least one criterion for particular patient data. The particular patient data can then be retrieved.

In an example, a subject or medical professional enters medical data from a biomarker assay into a webpage. The webpage transmits the data to a computer system or server, wherein the data is stored and processed. For example, the data can be stored in databases the computer systems. Processors in the computer systems can perform calculations comparing the input data to historical data from databases available to the computer systems. The computer systems can then store the output from the calculations in a database and/or communicate the output over a network to an output device, such as a webpage or email. After a user has received an output from the computer system, the user can take a course of medical action according to the output. For example, if the user is a physician and the output is a probability of cancer above a threshold value, the physician can then perform or order a biopsy of the suspected tissue.

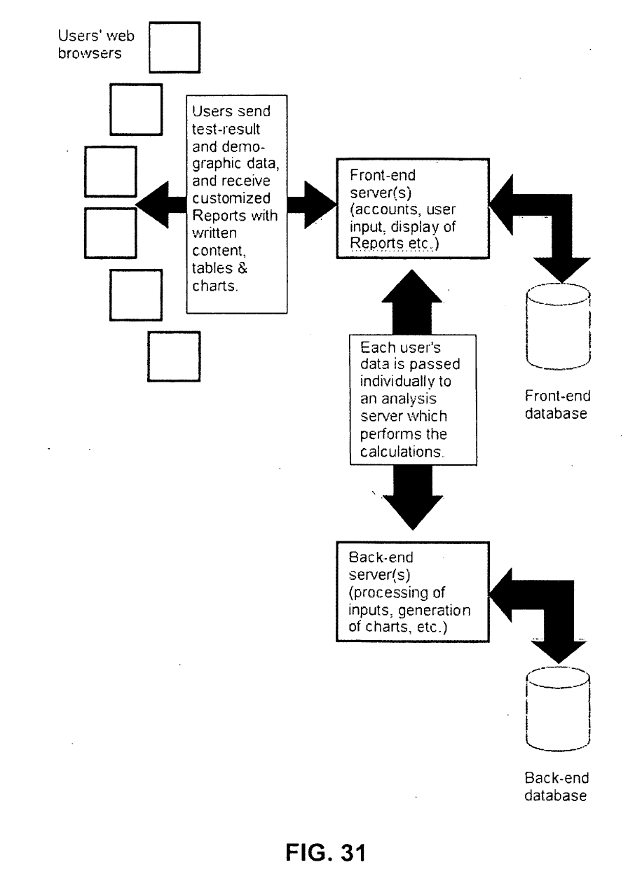

FIG. 31 demonstrates an example computer system of the invention. A set of users can use a web browser to enter data from a biomarker assay into a graphical user interface of a webpage. The webpage is a graphical user interface associated with a front end server, wherein the front end server can communicate with the user's input device (for example, a computer) and a back end server. The front end server can either comprise or be in communication with a storage device that has a front-end database capable of storing any type of data, for example user account information, user input, and reports to be output to a user. Data from each user (for example, biomarker values and subject profiles) can be then be sent to a back end server capable of manipulating the data to generate a result. For example, the back end server can calculate a probability that a subject has a medical condition using the data input by the user. A back end server can comprise historical data relating to a medical condition to be evaluated, or a plurality of medical conditions. The back end server can then send the result of the manipulation or calculation back to the front end server where it can be stored in a database or can be used to generate a report. The results can be transmitted from the front end server to an output device (for example, a computer with a web browser) to be delivered to a user. A different user can input the data and receive the data. In an embodiment, results are delivered in a report. In another embodiment, results are delivered directly to an output device that can alert a user.

In an embodiment, a method of the invention comprises obtaining a sample from a subject, wherein the sample contains a biomarker. The sample can be obtained by the subject or by a medical professional. Examples of medical professionals include, but are not limited to, physicians, emergency medical technicians, nurses, first responders, psychologists, medical physics personnel, nurse practitioners, surgeons, dentists, and any other obvious medical professional as would be known to one skilled in the art. The sample can be obtained from any bodily fluid, for example, amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In an example, the sample is obtained by a blood draw, where the medical professional draws blood from a subject, such as by a syringe. The bodily fluid can then be tested to determine the prevalence of the biomarker. Biological markers, also referred to herein as biomarkers, according to the present invention include without limitation drugs, prodrugs, pharmaceutical agents, drug metabolites, biomarkers such as expressed proteins and cell markers, antibodies, serum proteins, cholesterol, polysaccharides, nucleic acids, biological analytes, biomarker, gene, protein, or hormone, or any combination thereof: At a molecular level, the biomarkers can be polypeptide, glycoprotein, polysaccharide, lipid, nucleic acid, and a combination thereof.

Example biomarker assays include, but are not limited to, DNA assays, including DNA microarrays, Southern blots, Northern blots, ELISAs, flow cytometry, Western blots, PSA assays, and immunoassays. The information from the assay can be quantitative and sent to a computer system of the invention. The information can also be qualitative, such as observing patterns or fluorescence, which can be translated into a quantitative measure by a user or automatically by a reader or computer system. In an embodiment, the subject can also provide information other than biomarker assay information to a computer system, such as race, height, weight, age, gender, eye color, hair color, family medical history and any other information that may be useful to the user, as would be obvious.

Information can be sent to a computer system automatically by a device that reads or provides the data from a biomarker assay. In another embodiment, information is entered by a user (for example, the subject or medical professional) into a computer system using an input device. The input device can be a personal computer, a mobile phone or other wireless device, or can be the graphical user interface of a webpage. For example, a webpage programmed in JAVA can comprise different input boxes to which text can be added by a user, wherein the string input by the user is then sent to a computer system for processing. The subject may input data in a variety of ways, or using a variety of devices. Data may be automatically obtained and input into a computer from another computer or data entry system. Another method of inputting data to a database is using an input device such as a keyboard, touch screen, trackball, or a mouse for directly entering data into a database.

In general, in yet another aspect, a medical information system for delivering a probability of a medical condition of a subject to a user is provided including: a) an input device for obtaining biomarker values corresponding to a biomarker for a medical condition at least two different times from said subject, wherein said biomarker values form a biomarker trend; b) a processor in communication with said input device, wherein said processor uses said biomarker trend to calculate a posterior probability of said subject having said medical condition; and c) a storage unit in communication with at least one of the input device and the processor, wherein said storage unit includes at least one database including said biomarker values, said posterior probability, or a prior probability of said subject having said medical condition; and d) an output device in communication with at least one of said processor and said storage unit, wherein said output device transmits said posterior probability to a user.

The input device can be a graphical user interface of a webpage. The input device can be an electronic medical record. In an embodiment, a medical condition is prostate cancer. The biomarker can be PSA or fPSA.

In an embodiment, a processor and a storage unit can be part of a computer server. The processor can calculate a posterior probability that a subject has a medical condition by relating: a) a probability of observing said biomarker trend for an individual with said medical condition; b) a probability of observing said biomarker trend for an individual without said medical condition; and c) a prior probability that said subject has said medical condition.

An output device can be selected from a group including the following: a graphical user interface of a webpage, a print-out, and an email. The communication can be wireless communication.

In another embodiment, a system of the invention can further include a medical test for testing said subject for said medical condition. The medical test can be a PSA assay. In yet another embodiment, a system can further include a medical treatment for treating said subject for said medical condition. The medical treatment can be selected from a group including the following: a pharmaceutical, surgery, organ resection, and radiation therapy.

In an embodiment, a computer system of the invention comprises a storage unit, a processor, and a network communication unit. For example, the computer system can be a personal computer, laptop computer, or a plurality of computers. The computer system can also be a server or a plurality of servers. Computer readable instructions, such as software or firmware, can be stored on a storage unit of the computer system. A storage unit can also comprise at least one database for storing and organizing information received and generated by the computer system. In an embodiment, a database comprises historical data, wherein the historical data can be automatically populated from another database or entered by a user.

In an embodiment, a processor of the computer system accesses at least one of the databases or receives information directly from an input device as a source of information to be processed. The processor can perform a calculation on the information source, for example, performing dynamic screening or a probability calculation method of the invention. After the calculation the processor can transmit the results to a database or directly to an output device. A database for receiving results can be the same as the input database or the historical database. An output device can communicate over a network with a computer system of the invention. The output device can be any device capable delivering processed results to a user. Example output devices include, but are not limited to, a telephone, a wireless telephone, a mobile phone, a PDA, a flash memory drive, a light source, a sound generator, a fax machine, a computer, a computer monitor, a printer, an iPOD, and a webpage

An output of a computer system may assume any form, such as a computer program, webpage, or print-out. Any other suitable representation, picture, depiction or exemplification may be used.

Communication between devices or computer systems of the invention can be any method of digital communication including, for example, over the internet. Network communication can be wireless, ethernet-based, fiber optic, or through fire-wire, USB, or any other connection capable of communication as would be obvious to one skilled in the art. In an embodiment, information transmitted by a system or method of the invention can be encrypted by any method as would be obvious to one skilled in the art. In the field of medicine, or diagnostics, encryption may be necessary to maintain privacy of the data, as well as deter theft of information.

It is further noted that the systems and methods may include data signals conveyed via networks (for example, local area network, wide area network, internet), fiber optic medium, carrier waves, wireless networks for communication with one or more data processing or storage devices. The data signals can carry any or all of the data disclosed herein that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein. Other implementations may also be used, however, such as firmware or even appropriately designed hardware configured to carry out the methods and systems described herein.

The methods of the invention may be packaged as a computer program product, such as the expression of an organized set of instructions in the form of natural or programming language statements that is contained on a physical media of any nature (for example, written, electronic, magnetic, optical or otherwise) and that may be used with a computer or other automated data processing system of any nature (but preferably based on digital technology). Such programming language statements, when executed by a computer or data processing system, cause the computer system to act in accordance with the particular content of the statements. Computer program products include without limitation: programs in source and object code and/or test or data libraries embedded in a computer readable medium. Furthermore, the computer program product that enables a computer system or data processing equipment device to act in preselected ways may be provided in a number of forms, including, but not limited to, original source code, assembly code, object code, machine language, encrypted or compressed versions of the foregoing and any and all equivalents.

Information before, after, or during processing can be displayed on any graphical display interface in communication with a computer system (for example, a server). A computer system may be physically separate from the instrument used to obtain values from the subject. In an embodiment, a graphical user interface also may be remote from the computer system, for example, part of a wireless device in communication with the network. In another embodiment, the computer and the instrument are the same device.

An output device or input device of a computer system of the invention can include one or more user devices comprising a graphical user interface comprising interface elements such as buttons, pull down menus, scroll bars, fields for entering text, and the like as are routinely found in graphical user interfaces known in the art. Requests entered on a user interface are transmitted to an application program in the system (such as a Web application). In one embodiment, a user of user device in the system is able to directly access data using an HTML interface provided by Web browsers and Web server of the system.

A graphical user interface may be generated by a graphical user interface code as part of the operating system or server and can be used to input data and/or to display input data. The result of processed data can be displayed in the interface or a different interface, printed on a printer in communication with the system, saved in a memory device, and/or transmitted over a network. A user interface can refer to graphical, textual, or auditory information presented to a user and may also refer to the control sequences used for controlling a program or device, such as keystrokes, movements, or selections. In another example, a user interface may be a touch screen, monitor, keyboard, mouse, or any other item that allows a user to interact with a system of the invention as would be obvious to one skilled in the art.

In general, in yet another aspect, a method of taking a course of medical action by a user is provided including initiating a course of medical action based on a posterior probability delivered from an output device to said user.

The course of medical action can be delivering medical treatment to said subject. The medical treatment can be selected from a group consisting of the following: a pharmaceutical, surgery, organ resection, and radiation therapy. The pharmaceutical can include, for example, a chemotherapeutic compound for cancer therapy. The course of medical action can include, for example, administration of medical tests, medical imaging of said subject, setting a specific time for delivering medical treatment, a biopsy, and a consultation with a medical professional.

The course of medical action can include, for example, repeating a method described above.

A method can further include diagnosing the medical condition of the subject by said user with said posterior probability from said output device.

A system or method of the invention can involve delivering a medical treatment or initiating a course of medical action. If a disease has been assessed or diagnosed by a method or system of the invention, a medical professional can evaluate the assessment or diagnosis and deliver a medical treatment according to his evaluation. Medical treatments can be any method or product meant to treat a disease or symptoms of the disease. In an embodiment, a system or method initiates a course of medical action. A course of medical action is often determined by a medical professional evaluating the results from a processor of a computer system of the invention. For example, a medical professional may receive output information that informs him that a subject has a 97% probability of having a particular disease. Based on this probability, the medical professional can choose the most appropriate course of medical action, such as biopsy, surgery, medical treatment, or no action. In an embodiment, a computer system of the invention can store a plurality of examples of courses of medical action in a database, wherein processed results can trigger the delivery of one or a plurality of the example courses of action to be output to a user. In an embodiment, a computer system outputs information and an example course of medical action. In another embodiment, the computer system can initiate an appropriate course of medical action. For example, based on the processed results, the computer system can communicate to a device that can deliver a pharmaceutical to a subject. In another example, the computer system can contact emergency personnel or a medical professional based on the results of the processing. Courses of medical action a patient can take include self-administering a drug, applying an ointment, altering work schedule, altering sleep schedule, resting, altering diet, removing a dressing, or scheduling an appointment and/or visiting a medical professional. A medical professional can be for example a physician, emergency medical personnel, a pharmacist, psychiatrist, psychologist, chiropractor, acupuncturist, dermatologist, urologist, proctologist, podiatrist, oncologist, gynecologist, neurologist, pathologist, pediatrician, radiologist, a dentist, endocrinologist, gastroenterologist, hematologist, nephrologist, ophthalmologist, physical therapist, nutritionist, physical therapist, or a surgeon.

Medical professionals may take medical action when alerted by the methods of the invention of the medical condition of a subject. Examples of an alert include, but are not limited to, a sound, a light, a printout, a readout, a display, an alarm, a buzzer, a page, an e-mail, a fax alert, telephonic communication, or a combination thereof. The alert may communicate to the user the raw subject data, the calculated probability of the subject data.

The medical action can be based on rules imposed by the medical professional or the computer system. Courses of medical action include, but are not limited to, surgery, radiation therapy, chemotherapy, prescribing a medication, evaluating mental state, delivering pharmaceuticals, monitoring or observation, biopsy, imaging, and performing assays and other diagnostic tests. In an embodiment, the course of medical action may be inaction. Medical action also includes, but is not limited to, ordering more tests performed on the patient, administering a therapeutic agent, altering the dosage of an administered therapeutic agent, terminating the administration of a therapeutic agent, combining therapies, administering an alternative therapy, placing the subject on a dialysis or heart and lung machine, performing computerized axial tomography (CAT or CT) scan, performing magnetic resonance imaging (MRI), performing a colonoscopy, administering a pain killer, prescribing a medication. In some embodiments, the subject may take medical action. For example, a diabetic subject may administer a dose of insulin.

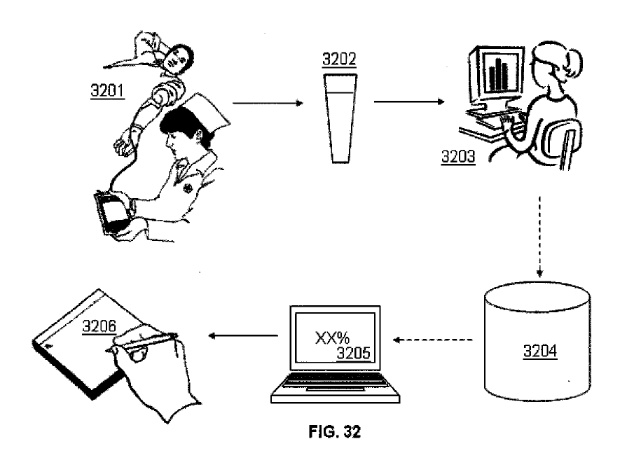

FIG. 32 illustrates a method of delivering a probability that a subject has a medical condition to a user and using the probability to take a course of medical action. A blood sample is drawn from a patient by a medical professional 3201. In other embodiments, any method of obtaining a biomarker values from a subject may be used as would be obvious to one skilled in the art, such as swabs and urine tests. In FIG. 32, the sample is assayed for a biomarker and biomarker values are generated 3202. As described herein, there may be many suitable methods for generating and obtaining biomarker values. The values can then input into a computer by a medical professional or other user 3203, such as the subject or an assistant. The data can then be processed 3204 by a method of the invention to calculate the probability that a subject has the medical condition. An output is generated and delivered to a user on a computer monitor 3205, for example, the output delivers the probability of a subject having a medical condition and is display on a personal computer or laptop of the subject's doctor. The output can also be delivered to the subject himself or to a different medical professional. In another embodiment, the output is delivered to a notification system, such as an alarm or another computer-based program. In FIG. 32, based on the output, a physician can take a medical action 3206 as described herein. In this example, the output initiates a medical professional writing a prescription 3206.

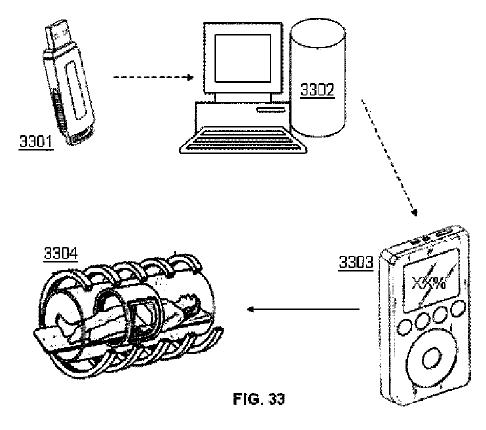

FIG. 33 illustrates a course of events related to the invention. Data regarding a biomarker corresponding to a medical condition from a patient are stored on a USB flash drive storage device 3301. Data are input into a computer system and data are processed by a calculation method of the invention 3302. For example, the computer system can be a server that receives data from multiple input devices and can distribute results of a calculation method to a plurality of output devices. In the example in FIG. 33, the results of the calculation method are a probability that a patient has a medical condition. The results delivered to the output device can also be suggestions of courses of medical action, reports based on the biomarker data, or warning or notification of the status of the patient and/or calculation. FIG. 33 also demonstrates displaying a probability of the medical condition of the subject on an output device such as an iPOD 3303. In this example, after reviewing the output, a user decides the course of medical action is a patient needs to obtain an MR image.

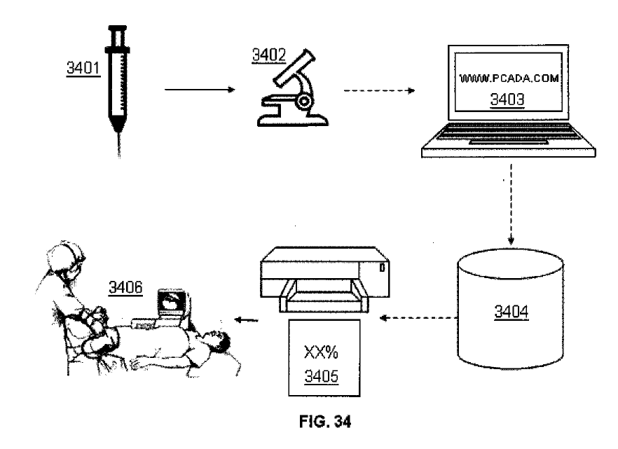

FIG. 34 illustrates another example practice of the invention. A sample is taken from a patient by a syringe 3401 and the sample is analyzed for a biomarker using a microscope 3402 to obtain a biomarker value corresponding to a medical condition. Using a graphical user interface 3403, such as a website, a user can enter the results of the analysis into the graphical user interface, or input device. The result of the biomarker analysis is transmitted from an input device, such as a laptop computer 3403 and the biomarker values are processed using a calculation method the invention in a server of the invention 3404. A probability of the subject from which the biomarker values were obtained is output to a printout from a printer 3405 to a user, such as the subject's physician. In this example, the physician may take a course of medical action that comprises delivering a medical treatment, such as performing an invasive surgical procedure 3406, such as a biopsy, based on results of the calculation.

In general, in yet another aspect, a method of delivering a probability of a medical condition of a subject to a user is provided including a) collecting biomarker values from a subject corresponding to a biomarker for a medical condition at least two different times, wherein the biomarker values at the at least two different times form a biomarker trend; b) exporting said biomarker trend for analysis, wherein said analysis includes: calculating a posterior probability that a subject has a medical condition by relating: i) a probability of observing said biomarker trend for an individual with said medical condition; ii) a probability of observing said biomarker trend for an individual without said medical condition; and iii) a prior probability that said subject has said medical condition; c) importing the results of said analysis to an output device; and d) delivering said posterior probability to a user with said output device.

It is to be understood that the exemplary methods and systems described herein may be implemented in various forms of hardware, software, firmware, special purpose processors, or a combination thereof. Preferably, a calculation method of the present invention is implemented in software as an application program tangibly embodied on one or more program storage devices. The application program may be executed by any machine, device, or platform comprising suitable architecture. It is to be further understood that, because some of the systems and methods depicted in the Figures are preferably implemented in software, the actual connections between the system components (or the process steps) may differ depending upon the manner in which the method is programmed. Given the teachings herein, one of ordinary skill in the related art will be able to contemplate or practice these and similar implementations or configurations of the present invention.

With respect to this disclosure, while examples have been used to disclose the invention, including the best mode, and also to enable any person skilled in the art to make and use the invention, the patentable scope of the invention is defined by claims, and may include other examples that occur to those skilled in the art. Accordingly the examples disclosed herein are to be considered non-limiting. As an illustration, it should be understood that for the processing flows described herein, the steps and the order of the steps may be altered, modified, removed, and/or augmented and still achieve the desired outcome.

### EXAMPLE 1

In an example, dynamic screening is a method for prostate cancer detection that uses trends in PSA, PSA velocity (PSAV), free PSA, free PSAV and prostate volume to estimate cancer risk. It was hypothesized that use of dynamic screening would detect cancers earlier than screening using a single PSA threshold, resulting in better long-term cancer control.

Exponential PSA trends were fit for men treated with radical prostatectomy (RP) who had at least 3 PSA tests over at least 2 years prior to RP with no PSA test 60% or greater from the trend (529 men in the SEARCH database from 1988 to 2007 and 304 from the Duke Prostate Center from 1989 to 2006). PSA values were determined when the cancer would have first been detected using dynamic screening and a PSA threshold. Using prostate weight, PSA at detection, and PSA trend, we estimated tumor volume (TV) if the cancer had been detected using 3 different screening strategies: 1) dynamic screening, 2) a single PSA threshold of >4.0 ng/ml, and 3) actual time of cancer detection. Using adaptations of published nomograms from Memorial Sloan Kettering (Stephenson et al, J Natl Cancer Inst; 17:715, 2006) and Johns Hopkins (Han et al, J. Urology; 169:517, 2003) adjusted to using TV rather than clinical or pathological stage, the 10-year risk of PSA recurrence was estimated. Gleason score was assumed not to change over time.

Dynamic screening resulted in the highest PSA free survival estimates followed by a PSA>4.0 cut-point with actual RP timing performing the worst as shown in Table 1. Average dynamic screening performance was nearly identical in both the SEARCH and Duke cohorts. Despite overall excellent performance, even with early detection using dynamic screening there remained a small proportion (<5%) of cancers in the Duke cohort that were predicted to have very poor long-term PSA free survival as shown in Table 1. These cancers had unusually large TV, which were not measured in the SEARCH cohort.

Therefore, dynamic screening using PSAV and PSA based on PSA trends leads to early detection, which would be predicted to lead to very high long-term PSA free survivals rates following RP relative to a standard PSA cut-point of 4.0.

TABLE 1
Ten Year Disease Freedom

| | Mean | | 50th | | 75th | | 90th | | 95th | | 98th | | 99th | |
|---|---|---|---|---|---|---|---|---|---|---|---|---|---|---|
| | VA | Duke | VA | Duke | VA | Duke | VA | Duke | VA | Duke | VA | Duke | VA | Duke |
| MSK Nomogram | | | | | | | | | | | | | | |
| Dynamic Screening | 98% | 98% | 100% | 99% | 99% | 97% | 96% | 92% | 92% | 84% | 87% | 65% | 74% | 48% |
| Static Screening (4.0) | 93% | 90% | 96% | 95% | 93% | 88% | 88% | 76% | 81% | 67% | 63% | 58% | 48% | 27% |
| Actual Treatment | 87% | 83% | 91% | 89% | 83% | 74% | 70% | 52% | 55% | 23% | 45% | 23% | 42% | 15% |
| Hopkins Tables | | | | | | | | | | | | | | |
| Dynamic Screening | 98% | 96% | 100% | 99% | 99% | 97% | 95% | 92% | 91% | 83% | 83% | 50% | 56% | 36% |
| Static Screening (4.0) | 92% | 89% | 95% | 95% | 92% | 87% | 82% | 73% | 70% | 60% | 51% | 37% | 30% | 32% |
| Actual Treatment | 83% | 79% | 90% | 89% | 79% | 69% | 55% | 39% | 36% | 27% | 26% | 9% | 22% | 6% |

### EXAMPLE 2

In another example, data were analyzed from 304 men diagnosed with prostate cancer and 9,380 men without diagnosed prostate cancer that were seen at the Duke Prostate Center from 1989 to 2006 who had a minimum of three PSA tests over at least a two year interval. Free PSA and prostate volume measurements were not considered because too few men had data for these variables. Static screening was evaluated by considering any PSA above a PSA threshold, such as 4.0, as a positive indication of cancer. Dynamic screening considered exponential PSA trends using PSA tests that were within 20% variation of the trend as variations >20% are much more likely to be caused by temporary conditions such as prostatitis than long term conditions such as progressing cancer. Results in excess of a calculated threshold based on PSA and PSA velocity trends and age were considered a positive indication of cancer. ROC curves were developed for the population as a whole and for three groups based on age (men in their 50s, 60s and 70s). AUCs were calculated in all cases. AUCs were also calculated for the entire population grouped by Gleason score (high=Gl 7 or greater and low—Gl 6 or lower) and tumor volume (0-1 cc, 1-3 cc, 3-5 cc and 5+cc). Full dynamic screening uses trends in Free PSA, as well as PSA trends used in this analysis.

In clinical use, a doctor with the help of full dynamic screening can use a process of elimination of possible benign conditions (BPH volume growth and prostatitis, both bacterial and non-bacterial) before using dynamic screening to conclude that cancer was probably progressing. For example, a jump in PSA combined with a drop in free PSA % are much more likely to be caused by bacterial prostatitis than progressing cancer. It was impossible to conduct this process of elimination on the retrospective data using free PSA trends. As a proxy for this process, AUCs were calculated for the high Gleason group and the four tumor volume groups as a function of the false positive rejection effectiveness percentage. One minus this percentage was multiplied by the number of false positives to simulate the number that would remain after the process of elimination using free PSA trends.

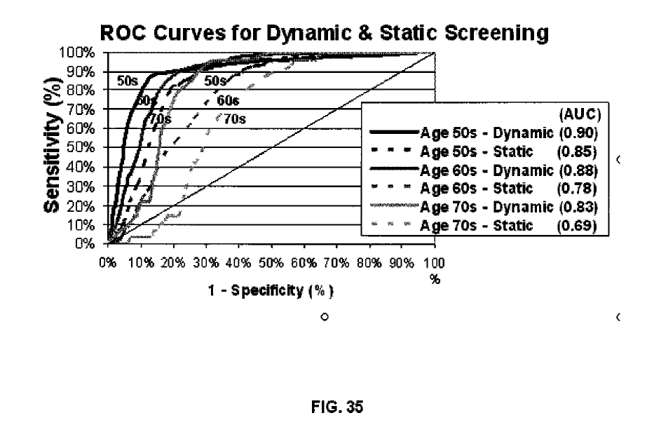

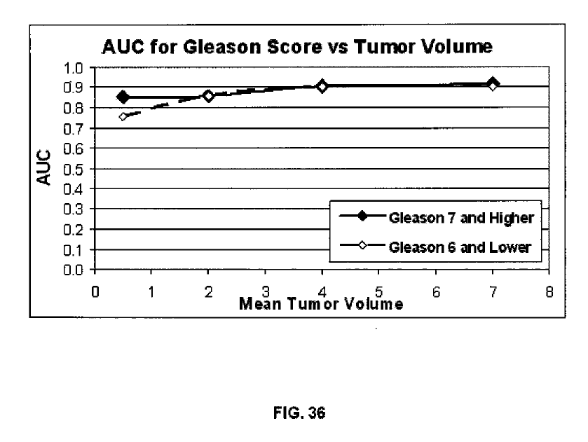

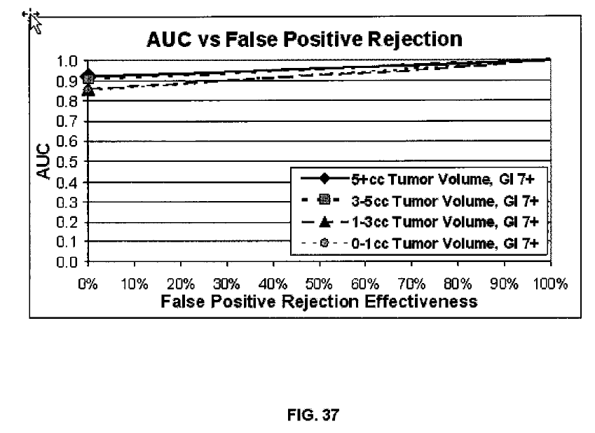

Dynamic screening delivered a higher AUC than static screening for the entire population (0.86 vs 0.74). AUCs were highest for younger men and declined with age as shown in FIG. 35. The improvement in AUC associated with dynamic screening relative to static screening increased with age. For the entire population, AUCs increased with mean tumor volume but did not vary substantially by Gleason group, except for the smallest tumor volumes as shown in FIG. 36. AUCs increased as the false positive rejection percentage increased as shown in FIG. 37. For example, if a doctor using a process of elimination with the help of dynamic screening based on free PSA trends can reject 75% of the remaining false positives then AUCs can increase to 0.98 for larger tumor volumes and 0.96 for smaller ones.

In conclusion, the simplest use of dynamic screening based only on PSA trends delivers higher sensitivity and specificity than does conventional static screening based on a PSA threshold. The performance gap increases for older men. Simple dynamic screening increases in performance for higher tumor volumes.

Dynamic screening can identify an increased probability of volume growth because it typically causes trend fPSAV % to increase above trend fPSA %. A doctor can confirm the hypothesis with an ultrasound measurement of prostate volume. Therefore, a significant proportion of false positives can be rejected by the use of dynamic screening with free PSA and volume measurements if necessary.

Dynamic screening delivers combinations of sensitivity and specificity for prostate cancer that are superior to conventional static screening using a single PSA threshold. Dynamic screening AUCs declined with age but remained better than static screening in all age ranges.

What is claimed is:

1. A method of delivering a probability that a subject has a medical condition to a user comprising:
a) calculating a posterior probability that a subject has a medical condition, wherein said subject has a biomarker trend, wherein said trend is formed by values corresponding to a biomarker for said medical condition obtained at least two different times from said subject, by relating:
i) a probability of observing said biomarker trend for an individual with said medical condition;
ii) a probability of observing said biomarker trend for an individual without said medical condition; and
iii) a prior probability that said subject has said medical condition; and
b) delivering said posterior probability to a user with an output device.

2. The method of claim 1, wherein said probability of observing said biomarker trend for an individual with said medical condition is calculated by comparing said biomarker trend to a historical probability distribution of historical biomarker trends of a population with said medical condition.

3. The method of claim 1, wherein said probability of observing said biomarker trend for an individual without said medical condition is calculated by comparing said biomarker trend to a historical probability distribution of historical biomarker trends of a population without said medical condition.

4. A method of delivering a probability that a subject has a medical condition to a user comprising:
a) calculating a posterior probability that a subject has a medical condition, wherein said subject has a biomarker value for said medical condition, by relating:
i) a probability of observing said biomarker value for an individual with said medical condition;
ii) a probability of observing said biomarker value for an individual without said medical condition; and
iii) a prior probability that said subject has said medical condition; and
b) delivering said posterior probability to a user with an output device.

5. The method of claim 4, wherein said probability of observing said biomarker value for an individual with said medical condition is calculated by comparing said biomarker value to a historical probability distribution of historical biomarker values of a population with said medical condition.

6. The method of claim 4, wherein said probability of observing said biomarker value for an individual without said medical condition is calculated by comparing said biomarker value to a historical probability distribution of historical biomarker values of a population without said medical condition.

7. A method of delivering a probability that a subject has a medical condition to a user comprising:
a) calculating a posterior probability that a subject has a medical condition, wherein said subject has a first biomarker value and a second biomarker value for said medical condition, wherein said second biomarker value is obtained after said first biomarker, by relating:
i) a probability of observing said second biomarker value for an individual with said medical condition;
ii) a probability of observing said second biomarker value for an individual without said medical condition;
iii) a probability of observing a biomarker rate of change for an individual with said medical condition, wherein said biomarker rate of change is the difference of biomarker values over time;
iv) a probability of observing said biomarker rate of change for an individual without said medical condition; and
v) a prior probability that said subject has said medical condition; and
b) delivering said posterior probability to a user with an output device.

8. The method of claim 7, wherein said probability of observing said biomarker rate of change for an individual with said medical condition is calculated by comparing said biomarker rate of change to a historical probability distribution of historical biomarker trends of a population with said medical condition.

9. The method of claim 7, wherein said probability of observing said biomarker rate of change for an individual without said medical condition is calculated by comparing said biomarker rate of change to a historical probability distribution of historical biomarker trends of a population without said medical condition.

10. The method of claim 7, wherein said probability of observing said biomarker value for an individual with said medical condition is calculated by comparing said biomarker value to a historical probability distribution of historical biomarker values of a population with said medical condition.

11. The method of claim 7, wherein said probability of observing said biomarker value for an individual without said medical condition is calculated by comparing said biomarker value to a historical probability distribution of historical biomarker values of a population without said medical condition.

12. The method of claim 1, 4 or 7, wherein said prior probability is calculated by comparing a profile of said subject to historical probabilities of said medical condition in an individual of a population;

13. The method of claim 1, 4, or 7 further comprising biomarker values from a second biomarker corresponding to said medical condition.

14. The method of claim 1, 4, or 7, wherein said medical condition is cancer.

15. The method of claim 14, wherein said cancer is prostate cancer.

16. The method of claim 1, 4, or 7, wherein said biomarker is fPSA or PSA.

17. The method of claim 1 or 7 further comprising removing a biomarker value from said biomarker trend that has a value outside a tolerance.

18. The method of claim 17, wherein said tolerance is determined by a historical biomarker trend representing said individual of a population with said medical condition.

19. The method of claim 17, wherein said tolerance is determined by a historical biomarker trend representing said individual of a population without said medical condition.

20. The method of claim 17, wherein said tolerance is set by said user.

21. The method of claim 17, wherein said tolerance is set automatically.

22. The method of claim 1, 4, or 7, wherein said calculating a posterior probability that a subject has a medical condition comprises at least one Monte Carlo simulation.

23. The method of claim 1, 4, or 7, wherein said calculating a posterior probability that a subject has a medical condition is carried out by a computer system.

24. The method of claim 23, wherein said computer system comprises a Monte Carlo calculation engine.

25. The method of claim 1, 4, or 7, wherein said user is selected from the group consisting of the following: said subject, a medical professional, a clinical trial monitor, and a computer system.

26. A method of taking a course of medical action by a user comprising initiating a course of medical action based on a posterior probability delivered from an output device to said user from a method of claim 1, 4, or 7.

27. The method of claim 26, wherein said course of medical action is delivering medical treatment to said subject.

28. The method of claim 27, wherein the medical treatment is selected from a group consisting of the following: a pharmaceutical, surgery, organ resection, and radiation therapy.

29. The method of claim 28, wherein said pharmaceutical comprises a chemotherapeutic compound for cancer therapy.

30. The method of claim 26, wherein the course of medical action comprises administration of medical tests.

31. The method of claim 26, wherein the course of medical action comprises medical imaging of said subject.

32. The method of claim 26, wherein the course of medical action comprises setting a specific time for delivering medical treatment.

33. The method of claim 26, wherein the course of medical action comprises a biopsy.

34. The method of claim 26, wherein the course of medical action comprises a consultation with a medical professional.

35. The method of claim 26, wherein the course of medical action comprises repeating a method of claim 1, 4, or 7.

36. The method of claim 1, 47 or 7 further comprising diagnosing the medical condition of the subject by said user with said posterior probability from said output device.

37. A computer readable medium comprising computer readable instructions, wherein the computer readable instructions instruct a processor to execute step a) of the method of claim 14, or 7.

38. The computer readable medium of claim 37, wherein the instructions operate in a software runtime environment.

39. A data signal that is transmitted using a network, wherein the data signal comprises said posterior probability calculated in step a) of the method of claim 1, 4, or 7.

40. The data signal of claim 39 further comprising packetized data that is transmitted through a carrier wave across the network.

41. A medical information system for delivering a probability of a medical condition of a subject to a user comprising:
a) an input device for obtaining biomarker values corresponding to a biomarker for a medical condition at least two different times from said subject, wherein said biomarker values form a biomarker trend;
b) a processor in communication with said input device, wherein said processor uses said biomarker trend to calculate a posterior probability of said subject having said medical condition;
c) a storage unit in communication with at least one of the input device and the processor, wherein said storage unit comprises at least one database comprising said biomarker values, said posterior probability, or a prior probability of said subject having said medical condition; and
d) an output device in communication with at least one of said processor and said storage unit, wherein said output device transmits said posterior probability to a user.

42. The system of claim 41, wherein said input device is a graphical user interface of a webpage.

43. The system of claim 41, wherein said input device is an electronic medical record.

44. The system of claim 41, wherein said medical condition is prostate cancer.

45. The system of claim 41, wherein said biomarker is PSA or fPSA.

46. The system of claim 41, wherein said processor and said storage unit are part of a computer server.

47. The system of claim 41, wherein said processor calculates a posterior probability that a subject has a medical condition by relating:
a) a probability of observing said biomarker trend for an individual with said medical condition;
b) a probability of observing said biomarker trend for an individual without said medical condition; and
c) a prior probability that said subject has said medical condition.

48. The system of claim 41, wherein said output device is selected from a group consisting of the following: a graphical user interface of a webpage, a print-out, and an email.

49. The system of claim 41, wherein said communication is wireless communication.

50. The system of claim 41 further comprising a medical test for testing said subject for said medical condition.

51. The system of claim 51, wherein said medical test is a PSA assay.

52. The system of claim 41 further comprising a medical treatment for treating said subject for said medical condition.

53. The method of claim 53, wherein the medical treatment is selected from a group consisting of the following: a pharmaceutical, surgery, organ resection, and radiation therapy.

54. A method of delivering a probability of a medical condition of a subject to a user comprising:
a) collecting biomarker values from a subject corresponding to a biomarker for a medical condition at least two different times, wherein the biomarker values at the at least two different times form a biomarker trend;
b) exporting said biomarker trend for analysis, wherein said analysis comprises:
calculating a posterior probability that a subject has a medical condition by relating: i) a probability of observing said biomarker trend for an individual with said medical condition; ii) a probability of observing said biomarker trend for an individual without said medical condition; and iii) a prior probability that said subject has said medical condition;
c) importing the results of said analysis to an output device; and
d) delivering said posterior probability to a user with said output device.
